(19) United States
(12) Patent Application Publication
(10) Pub. No.: US 2004/0157268 A1
(43) Pub. Date: Aug. 12, 2004

(54) Conformational assays to detect binding to membrane spanning, signal-transducing proteins

(76) Inventors: Brian K. Kobilka, Palo Alto, CA (US); Pejman Ghanouni, Menlo Park, CA (US); Tae Weon Lee, Palo Alto, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/692,071

(22) Filed: Oct. 22, 2003

Related U.S. Application Data
(63) Continuation of application No. PCT/US02/13250, filed on Apr. 24, 2002.
Application No. PCT/US02/13250 is a continuation-in-part of application No. 09/935,061, filed on Aug. 21, 2001.
(60) Provisional application No. 60/286,250, filed on Apr. 24, 2001.

(51) Int. Cl. G01N 33/53; G01N 33/567

(52) U.S. Cl. 435/7.2

(57) ABSTRACT

The present invention provides methods and compositions for detection of compounds that have activity in modulating activity of membrane-spanning, signal-transducing (MSST) proteins, e.g., agonists, and antagonists. The detection method is based upon detection of a conformational change in a MSST protein upon interaction with a ligand. Conformational change of the MSST protein upon ligand interaction is accomplished by modifying the MSST protein to comprise a conformationally sensitive detectable probe, so that ligand interaction that results in a conformational change in the MSST protein is detected by a change in detectable signal from the detectable probe. The conformationally sensitive detectable probe can be a chemical label (e.g., a fluorophore) or moiety integral to the protein (e.g., a protease cleavage site, or immunodetectable moiety). The conformational assays of the invention provide for high-throughput screening.

### CROSS-REFERENCE TO RELATED APPLICATION

This application 1) is a continuation of International Application No. PCT/US02/13250, filed Apr. 24, 2002, which application was published in English; 2) is a continuation-in-part- of earlier filed U.S. application Ser. No. 09/935,016, filed Aug. 21, 2001; and 3) claims the benefit of earlier-filed U.S. provisional application serial No. 60/286,250, filed Apr. 24, 2001, each of which applications are incorporated herein by reference in their entireties.

### GOVERNMENT RIGHTS

The United States Government may have-certain rights in this application-pursuant to Grant 5RO1.NS28471.

### FIELD OF THE INVENTION

This invention relates to methods and compositions for detection of activity of a membrane spanning, signal-transducing protein, and methods of screening for ligands, and other proteins that affect processes regulated by such proteins.

### BACKGROUND OF THE INVENTION

Despite their diverse physiologic roles, many membrane spanning proteins involved in signal transduction share structural features. These shared structural features include one or more transmembrane domains, which position the protein within a cellular membrane. Additional shared structural features include at least one extracellular domain, which, along with the transmembrane domains, may be involved in interactions with a ligand(s) (e.g., extracellular agonists and antagonists), and intracellular domains, which facilitate transduction of a signal depending on the presence of a ligand. In addition, these membrane-spanning, signal-transducing proteins (or “MSST” proteins) share a common activation mechanism, which involves a conformational change in one or more transmembrane domains upon interaction with ligand.

For example, although they are diverse in their function and activity, the majority of G protein coupled receptors (GPCRs) are composed of seven transmembrane domains, which are connected by intracellular and extracellular loops. GPCRs share a common activation mechanism. Briefly, agonists induce conformational changes in receptors, which then stimulate heterotrimeric GTP-binding proteins (G proteins). Activated G proteins influence cellular physiology by modulating specific effector enzymes and ion channels involved in cardiovascular, neural, endocrine, and sensory signaling systems (see, e.g., Strader et al., Annu Rev Biochem 63:101-32 (1994)).

The actions of many extracellular signals are mediated by the interaction of guanine nucleotide-binding regulatory proteins (G proteins) and G-protein coupled receptors (GPCRs). Individual GPCRs activate particular signal transduction pathways through binding to G proteins, which in turn transduce a signal to the cell to elicit a response from the cell. GPCRs are known to respond to numerous extracellular signals, including neurotransmitters, drugs, hormones, odorants and light. The family of GPCRs has been estimated to include several hundred members, fully more than 1.5% of all the proteins encoded in the human genome. The GPCR family members play roles in regulation of biological phenomena involving virtually every cell in the body. The sequencing of the human genome has led to identification of numerous GPCRs; although the ligands and functions of many of these GPCRs are known, a significant portion of these identified receptors are without known ligands. These latter GPCRs, known as “orphan receptors”, also generally have unknown physiological roles.

Channels and transporter proteins also fall within the class of MSST proteins which share the structural features and mechanism of action discussed above. Channels function as pores or holes traversing the lipid bilayer of a cell, which, in a regulated manner, selectively facilitate the movement of solutes or water across cell membranes. They share the common function of transporting solutes and water across cell membranes; unsurprisingly, they share common structural features, including multiple transmembrane domains and critical pore-loop structures. Channels are responsible for generating and propagating electrical impulses in excitable tissues in the brain, heart, and muscle, and for setting the membrane potential of excitable and non-excitable cells. Channels also provide a pathway for communication between and within cells (see, e.g., Kanner, B. I., J. exp. Biol. 196: 237-249 (1994), and Nelson, N., J. Neurochem. 71: 1785-1803 (1998)).

Ion channels alter their activity in response to transmitter actions and the metabolic state of the cell so as to modulate cellular excitability. Mechanistically, ion channels may be opened by changes in the voltage of the membrane in which they reside (voltage-gated) or by the presence of neurotransmitter (ligand-gated). As a general mechanism, ion channels recognize specific ligands or detect voltage changes, transduce this binding or electrical changes into propagated conformational changes which open or close (i.e. gate) the channel, and select and conduct specific ions through a transient opening through the membrane. As ions flow through it down their electrochemical gradients; the potential across the membrane changes, and molecules within the target cell respond. The neurotransmitters that activate some ion channels are removed by high-affinity neurotransmitter transporter proteins also present near the sites of neurotransmitter release.

Transporter proteins, such as those used for transport of dopamine, GABA, catecholamines and serotonin across a membrane, share a common topology characterized by twelve transmembrane segments. Functionally, these proteins are located in the membranes of the pre-synaptic cell or in the membranes of nearby glial cells. The transport cycle of these transporter proteins couple sodium binding to the transporter to substrate binding in the extracellular environment; this binding triggers a conformational change that releases the substrate and sodium within the intracellular environment. The reuptake of neurotransmitter mediated by these proteins is critical to quickly limiting the time and scope of neurotransmitter release, thereby regulating synaptic efficacy.

Many available therapeutic drugs in use today target membrane-spanning, signal-transducing proteins. For example, identification of compounds that modulate GPCR activity are of interest, since GPCRs mediate various vital physiological responses, including vasodilation, heart rate, bronchodilation, endocrine secretion, and gut peristalsis. See, eg., Lefkowitz et al., Ann. Rev. Biochem. 52:159 (1983); Gilman, A. G. (1987) Annu. Rev. Biochem 56: 615-649; Hamm, H. E. (1998) JBC 273: 669-672; Ji, T. H. (1998) JBC 273: 17229-17302; Kanakin, T. (1996) Pharmacological Review, 48:413-463; Gudermann T. and Schultz, G. (1997), Annu. Rev. Neurosci., 20: 399-427. In fact, it has been estimated that more than 50% of the drugs in use clinically in humans at the present time are directed at GPCRs, including the adrenergic receptors (ARs). For example, ligands to beta ARs are used in the treatment of anaphylaxis, shock, hypertension, hypotension, asthma and other conditions. Similarly, identification of compounds that modulate activity of ion channels and transporter proteins are of interest, since these proteins play vital roles in basic physiologic processes including regulation of locomotor activity, cognitive functions, and neuroendocrine systems. See, e.g., Lerche et al., Am. J. Med. Genet. 106(2):146-59, Cooper, Epilepsia, 42 Suppl. 5:49-54, Tassonyi et al., Brain Res Bull 57(2):133-50, Langan, Curr Cardiol Rep 1(4):302-7, Noll et al., Cardiology 89 Suppl1:10-15, Opie, L. H Prog. Cardiovasc. Dis. 38(4):273-90, Rothman et al., Pharmacol. Biochem. Behav. 71(4):825-36, Frazer et al., Int. J. Neuropsychopharmacol. 2(4):305-320, Lesch, K. P., J. Affect. Disord. 62 (1-2):57-76, Iversen, L. Mol. Psychiatry, 5(4):357-62, Chamey, D. S., J. Clin. Psychiatry. 59 Suppl 14:11-4, Owens et al., Clin. Chem. 40(2):288-95, Fuller, R. W. J. CLin. Psychiatry 52 Suppl:52-7, Klein et al., Jpn. J. Pharmacol. 70 (1):1-15, Costa, E. Neuropsychopharmacology 2(3):167-74, Ticku et al., Life Sci. 33(24):2363-75, Tallman et al., Science 207(4428):274-81. Drugs that act on ion channel proteins are used to induce anesthesia, and treat epilepsy, cardiac arrhythmias, coronary artery disease and hypertension. Drugs that act on ligand gated ion channels and transporters are used to treat neuropsychiatric disorders such as anxiety, depression, attention deficit disorder, and schizophrenia.

Since MSST proteins are critical targets for therapeutics, there is a need in the art for fast, effective and reproducible methods for identifying agonists, antagonists and inverse agonists that modulate signaling mediated by MSST proteins. In general, three different approaches to identify such compounds have been described. A first approach for identification of agents that activate a MSST protein, such as a GPCR, is based on the ability of the compound to bind to the protein, e.g., as in a competitive binding assay. Binding assays measure the ability of a molecule (e.g., candidate agent) to displace the binding of a known ligand to the receptor. They are limited by the availability of such ligands and are therefore not useful for MSST proteins for which the ligand is not known e.g., orphan GPCRs.

A second approach is to screen candidate agents for the ability to activate function of a MSST protein, e.g., a functional assay. Signaling assays measure the ability of ligands to activate components of a signal transduction cascade, such as G protein or second messenger activation in the case of GPCRs (Tota et al. (1990) Mol Pharmacol 37(6), 996-1004; Selley, et al. (1997) Mol Pharmacol 51(1), 87-96; Krumins, et al. (1997) Mol Pharmacol 52(1), 144-54; 4. Perez, et al. (1996) Mol Pharmacol 49(1), 112-22). These conventional assays are best suited for detecting agonists. The effectiveness of this type of assay is somewhat dependent on the specificity of the interaction between the MSST protein and its downstream effectors, e.g., specificity of G protein coupling with the GPCR. More importantly, this type of assay requires that the downstream effector and/or the second messenger be known. In the case of channels and transporters, these functional assays are not amenable for high through put screening.

A third approach involves detection of conformational changes. Several biophysical studies on the β₂AR and rhodopsin have demonstrated conformational changes in TM6 or the attached intracellular loop 3 (IC3) region upon ligand activation (Sheikh, et al. (1996) Nature 383(6598), 347-50; Altenbach, et al. (1996) Biochemistry 35(38), 12470-8; Farrens, et al. (1996) Science 274(5288), 768-70; Gether, et al. (1997) Embo J 16(22), 6737-47). However, the techniques in these studies require labeling of multiple sites in the receptor and/or are not amenable to high throughput screening-(e.g., the assays do not provide a large enough difference in detectable signal to make the assay useful in high throughput screening). Other conventional techniques focus upon the use of surface plasmon resonance techniques, which are tedious, time consuming, and not easily adapted to high-throughput screening.

Currently available assay technologies to measure ion channel and transporter activity in a biological membrane are voltage-clamping of membrane patches (referred to as patch-clamping), efflux assays using fluorescent voltage-sensitive probes and fluorescent ion-sensitive dyes, and influx assays using radiolabeled or fluorescently labeled substrate analogues. In addition to the above functional assays, the radioligand binding assay is a conventional method to detect compound activity to ion channels. The most popular ion channel assay is patch clamping, which provides high quality and physiologically relevant data of channel function at the single cell (eg. oocytes). However, setting up patch clamping experiments is a complicated process requiring highly trained personnel to avoid experimental variations, and the process is very low throughput. For fluorescence-based high throughput assays, FLIPRTM (fluorometric Imaging Plate Reader, Molecular Devices, Sunnyvale, Calif.) and VIPRTM (Voltage Ion probe reader; Aurora Biosciences, San-Diego, Calif.) are the current leading technologies. However, voltage-sensor dyes show a lower kinetics that do not mirror the physiologic behavior of ion channels. Although dye cost is relatively inexpensive, the instrument itself is very expensive. Assays using radioisotopes (e.g., 86Rb+ for K+ channels) to trace the cellular influx and efflux of specific ions are much higher throughput than that of patch clamp but face the challenges and costs of handling large amounts of radioactive materials (Fox, S., Cambridge Healthtech Institute's 8th Annual High throughput Technologies, Philadelphia, Pa., Schroeder, K., Society for Biomolecular Screening 7th Annual Conference, Baltimore, Md., Terstappen, G. Anal. Biochem. 272, 149-155, Gonzales, J. and Tsien R. Chem. Biol. 4, 269-277, Cronk, D. et al. Society for Biomolecular Screening 7th Annual Conference, Baltimore, Md., Gonzales J. et al. Drug Discov. Today 4, 431-439, Denyer, J. et al. Drug Discov. Today 3, 323-332, Lachnit, W. et al. Drug Discov. Today 6, S17-18, .Xu, et al. Drug Discov. Today 6,1278-1287, Farina, J. et al. Anal. Biochem. 295, 138-142).

There is a need in the field for assays for detection of candidate agents that modulate activity of MSST proteins, and which can be readily adapted to high-throughput screening of candidate agents. The present invention addresses this need.

### SUMMARY OF THE INVENTION

The present invention provides methods and compositions for detection of molecules that have activity in modulating activity of membrane-spanning, signal-transducing (MSST) proteins, e.g., agonists, and antagonists. The detection method is based upon detection of a conformational change in a membrane-spanning, signal-transducing protein upon interaction with a ligand. Conformational change of the MSST protein upon ligand interaction is accomplished by modifying the MSST protein to comprise a conformationally sensitive detectable probe, so that ligand interaction that results in a conformational change in the MSST protein is detected by a change in detectable signal from the detectable probe. The conformationally sensitive detectable probe can be a chemical label (e.g., a fluorophore) or moiety integral to the protein (e.g., a protease cleavage site, or immunodetectable moiety). The conformational assays of the invention provide for high-throughput screening.

Thus, in one aspect the invention features methods for identifying agents that modulate activity of a MSST protein, where the method comprises contacting a MSST protein with a candidate agent. The MSST protein having a conformationally-sensitive detectable probe positioned on or within a conformationally sensitive region of the MSST protein such that interaction of the MSST protein with an agonist or antagonist causes a conformational change in the conformationally sensitive region and a change in a detectable signal of the conformationally sensitive detectable probe. A detectable signal of the conformationally sensitive detectable probe resulting from contacting of the candidate agent is detected. Detection of a change in a level of the detectable signal in the presence of the candidate agent relative to a control level of detectable signal indicates the candidate agent modulates activity of the MSST protein. The control can be either a positive control (e.g., a level of detectable signal caused by a known MSST protein agonist or antagonist) or a negative control (e.g., a level of detectable signal in the absence of candidate agent or a level of detectable signal in the presence of an agent that is known not to modulate activity of the MSST protein).

In exemplary embodiments, the conformationally-sensitive detectable probe is a detectable chemical label attached to an amino acid residue of the conformationally sensitive region. In other exemplary embodiments, the conformationally-sensitive detectable probe is an integral detectable moiety, which may be a protease cleavage site or an immunodetectable probe.

Where the probe is a protease cleavage site, the detectable signal is a protease cleavage product. In some embodiments, the conformationally-sensitive detectable probe comprises two protease cleavage sites, which cleavage sites flank a detectable polypeptide so that cleavage of the cleavage sites results in release of the detectable polypeptide, and wherein the detectable signal is the detectable polypeptide.

Where the probe is an immunodetectable epitope, the detectable signal can be present on a primary antibody that specifically binds the epitope or on a secondary antibody that specifically binds the primary antibody.

In further exemplary embodiments, the conformationally sensitive region is in an intracellular loop, an extracellular loop, an N-terminal domain, or a C-terminal domain of the MSST protein.

In still further exemplary embodiments of features and embodiments above, the MSST protein is a G protein coupled receptor (GPCR), an ion channel, or a transporter protein.

In one embodiment, the MSST protein is a G-protein coupled receptor (GPCR), and the conformationally sensitive region is an intracellular loop, an extracellular-loop, an N-terminal domain, or a C-terminal domain of the GPCR.

In a further exemplary embodiment, the conformationally sensitive region of the GPCR is a third intracellular loop of the GPCR, and the conformationally sensitive detectable probe is a detectable chemical label attached to one or more amino acid residues within the third intracellular loop so that a conformational change in the GPCR due to interaction with an agonist or antagonist causes a change in the detectable signal of the detectable probe. In a specific exemplary embodiment, the detectable chemical label is attached to an amino acid residue corresponding to amino acid residue at position 265 in a β2-adrenergic receptor.

In another exemplary embodiment, the MSST protein is a GPCR, the conformationally sensitive detectable probe is a protease cleavage site, and the detectable signal is a protease cleavage product. The protease cleavage product can be an N-terminal fragment of the GPCR, a C-terminal fragment of the GPCR.

The invention also features apparatuses for detecting a molecule that modulates activity of a MSST protein, where the apparatus comprises a (MSST) protein in any of the above-described features and embodiments, and an immobilization phase to which the MSST protein is attached.

The invention also features kits for use in screening a candidate agent, where the kit comprises a MSST protein as described in the above features and specific exemplary embodiments of the invention. In exemplary embodiments, the MSST protein of the kit is attached to an immobilization phase.

The present invention provides rapid and sensitive bioassays for evaluating new agonists, antagonists and/or inverse agonists for MSST protein, such as GPCRs, ion channels, and transporter proteins.

The invention also provides methods for identification of ligands for MSST proteins, and can be used to identify MSST proteins involved in different biological processes, including disease.

The invention can also be used to detect the presence of a particular ligand in a sample, e.g., the presence of a drug such as an opioid.

An advantage of one embodiment of the invention, in which the conformationally sensitive probe is an integral moiety (e.g., an amino acid sequence that defines, for example, a protease cleavage site or an immunodetectable epitope), is that the assays can be performed using membranes, which increases both the ease of performing the assay and the efficacy of the assay.

Another advantage is that assays of the invention allow high throughput screening of MSST protein activity.

Yet another advantage of the invention is that it allows for determination of the affinity and efficacy of a ligand for a MSST protein.

Still another advantage of the invention is that, when provided in an array format, the invention can provide for determination of ligand specificity with a specific MSST protein on the array.

These and other advantages and features of the invention will become apparent to those persons skilled in the art upon reading the details of the apparatus and assays as more fully described below.

### BRIEF DESCRIPTION OF THE DRAWINGS

FIGS. 1A-1C are schematic diagrams of the secondary structure of β₂AR illustrating the fluorescein maleimide (FM) labeling site at Cys265.

FIG. 1A illustrates the position of the 13 cysteines (C in a circle) in the β2AR, yet only Cys265 is labeled with the relatively large, polar fluorophore FM under the conditions described in the Methods below. Cysteine residues are indicated by circles; aspartic acid residues by D in a circle; phenylalanine by F in a circle; and serine by S in a circle. Cys106, Cys184, Cys190, and Cys191 have been shown to be disulfide bonded and Cys341 is palmitoylated. Cys378 and Cys406 in the carboxyl terminus form a disulfide bond during purification. Labeling specificity was confirmed by peptide mapping and mutagenesis of potential reactive cysteines (data not shown). The sites of peptide cleavage by Factor Xa (line) and cyanogen bromide (black dots) are shown.

FIG. 1B is a schematic of transmembrane helices 5 and 6 and the connecting intracellular loop 3 (IC3). The location of the fluorescein maleimide (F) site is highlighted. Fluorescence quenchers (squares) localized to either the aqueous milieu, the micellar environment, or to the base of TM5 (oxyl-N-hydroxysuccinimide bound to Lys224, large square) were used to monitor conformational changes around Cys265.

In FIG. 1C, cylinders representing the seven transmembrane helices of the β2AR as viewed from the cytoplasmic side of the membrane; arranged according to the crystal structure of rhodopsin in the inactive state. In the inactive receptor, FM on Cys265 is predicted to point toward the cytoplasmic extensions of transmembranes 3, 5, and 6. Also shown is the predicted position of the quencher oxyl-NHS on Lys224 (square).

FIGS. 2A-2B illustrate the effect of agonists and partial agonists on fluorescence intensity of FM-β₂AR.

In FIG. 2A, the change in intensity of FM-β₂AR in response to the addition of the full agonist (−)-isoproterenol (ISO) and the strong partial agonist epinephrine (EPI) was reversed by the neutral antagonist (−)-alprenolol (ALP). FIG. 2B illustrates the agonist and partial agonist effects on the intensity of FM-β2AR compared with an assay of biological efficacy (GTPγS binding).

FIGS. 3A-3B illustrate the response of FM-β2AR to agonist in the presence of potassium iodide or Oxyl-NHS. FIG. 3A is a Stern-Volmer plots of KI quenching of FM-labeled β2AR. FIG. 3B shows the effect of quenchers KI and Oxyl-NHS on the magnitude of the ISO-induced decrease in fluorescence.

FIGS. 4A-4D provide a comparison of effects of quenchers localized to the micelle on the response of FM-β2AR to (−)-isoproterenol.

FIG. 4A is a schematic depicting the structure of CAT-16 and 5-doxyl stearate (5-DOX), as well as the putative location of these quenching groups in the micelle. The quenching group on 5-DOX is located within the hydrophobic core of the micelle.

FIG. 4B is a Stern-Volmer plot depicting the extent of quenching of FM-β2AR by increasing concentrations of CAT-16 or 5-DOX.

FIG. 4C illustrates the differing effects of CAT-16 and 5-DOX on agonist-induced fluorescence change of FM-β2AR. The extent of response to (−)-isoproterenol is presented as a % control ISO response, calculated as in FIG. 3.

FIG. 4D is an example of the experiments used to generate the ratios in FIG. 4C.

FIGS. 5A and 5B are schematics showing agonist-induced conformational changes in TM6. The model represents TM 3, 5, and 6 as viewed from the cytoplasmic surface of the receptor arranged according to the crystal structure of rhodopsin. FM on Cys265 is indicated by the circle; oxyl-NHS on Lys224 is indicated by the square. The results from quenching experiments can best be explained by either a clockwise rotation of TM6 (FIG. 5A) and/or tilting of TM6 (FIG. 5B) toward TM5 during agonist-induced activation of the receptor.

FIG. 6A is a schematic diagram of the secondary structure of β2 AR illustrating the fluorescein maleimide (FM) labeling site at Cys265. Amino acids in dark circles have been shown to be important for agonist binding.

FIG. 6B is a graph showing the effect of the full agonist (−)-isoproterenol (ISO) on fluorescence intensity of FM-β2AR. Purified, detergent-solubilized β2-AR was labeled with FM at Cys265 and examined by fluorescence spectroscopy. Change in intensity of FM-b2 AR in response to the addition of ISO followed by the reversal by the neutral antagonist (−)-alprenolol (ALP).

FIG. 7 is a graph showing the effect of drugs on fluorescence lifetime distributions of FM-β2 AR. Fluorescence lifetimes were determined by phase modulation and lifetime distributions of FM-β2 AR were calculated in the absence of ligand, with the neutral antagonist ALP, or in the presence of the full agonist ISO. The mean lifetime and the full width at half maximum for the distributions are: No Ligand τ=4.21±0.01 nsec, FWHM=1.1±0.1, χ²=2.8; ALP: τ=4.21±0.01 nsec, FWHM=0.7±0.2, χ²=2.9; ISO: τ_{LONG}=4.36±0.08 nsec, FWHM_{LONG}=0.5±1.1, τ_{SHORT}=0.76±0.33 nsec, FWHM_{SHORT}=1.7±1.2, χ²=3.2.

FIGS. 8A and 8B are graphs showing the comparison of the effects of full and partial agonists on the fluorescence lifetime distributions of FM-β2 AR. In FIG. 8A the effect of the full agonist ISO and partial agonists SAL and DOB on the lifetime distributions of FM-β2 AR are compared. FIG. 8B provides an expanded view of the short lifetime distributions shown in FIG. 8A. The mean lifetime and the full width at half maximum for the new distributions are: SAL: τ_{LONG}=4.37±0.04 nsec, FWHM_{LONG}=0.7±0.3, τ_{SHORT}=1.93±0.24 nsec, FWHM_{SHORT}=0.7±0.3, χ²=2.1; DOB: τ_{LONG}=4.38±0.01 nsec, FWHM_{LONG}=0.4±0.4, τ_{SHORT}=1.78±0.01, FWHM_{SHORT}=0.9±0.6, χ²=2.0.

FIGS. 9A-9B are diagrams of the two-state model of GPCR activation. In FIG. 9A, R is the inactive conformation and R* is the active conformation capable of activating the G protein. The equilibrium between R and R* is influenced differently by agonists (ISO) and partial agonists (DOB). The width of the arrows reflects the rate constant. FIG. 9B is a diagram of a multistate model of GPCR activation. The agonist ISO and the partial agonist DOB both induce an intermediate state R′, as well as distinct G protein activating conformations R* and R^{x}, respectively. The neutral antagonist ALP induces a conformation R^{o} that is functionally equivalent to R at activating the G protein Gs, but can be distinguished from R by susceptibility to digestion by proteases.

FIG. 10 is schematics showing a GPCR having a protease cleavage site positioned so that ligand binding results in a conformational change that alters the accessibility of the protease cleavage site to protease cleavage (i.e., the protease site is either more or less accessible to protease cleavage as a result of a ligand-induced conformational change).

FIG. 11A is a schematic showing a modified GPCR (β2-adrenergic receptor) having a Flag epitope, and an introduced cleavage site (TEV protease) as a conformationally sensitive probe in the third intracellular loop, between transmembrane domains 6 and 7

FIG. 11B is a photograph of a Western blot showing agonist dependent cleavage of a TEV protease site in the β2 adrenergic receptor. Insect cell membranes expressing the modified β2 adrenergic receptor shown in FIG. 11A were used. Intact and TEV-cleaved β2 adrenergic receptor were detected with M1 Flag antibody which recognizes the amino terminal Flag epitope. Membranes were treated with the agonist isoproterenol (ISO) and TEV protease (TEV) as indicated in the figure. Isoproterenol treatment increases the ability of TEV protease to cleave the β2 adrenergic receptor.

FIG. 11C is a plot of the ratio of TEV cleaved to uncleaved β2 adrenergic receptor in the presence or absence of the agonist isoproterenol in the experiment of FIG. 11B.

FIG. 12 is a schematic showing the amino acid sequence of β2-adrenergic receptor (SEQ ID NO:6) and modifications that can be made within the second intracellular loop (SEQ ID NO:8) or within the third intracellular loop (SEQ ID NO:10) to insert a protease cleavage site (exemplified by tobacco etch virus (TEV)) that can serve as a conformationally sensitive probe for ligand binding.

FIG. 13 is a schematic showing the DNA (SEQ ID NO:5) and amino acid (SEQ ID NO:6) sequences of the of the β2-adrenergic receptor.

FIG. 14 is a schematic showing the DNA (SEQ ID NO:7) and amino acid (SEQ ID NO:8) sequences of a β2-adrenergic receptor modified to contain a TEV protease cleavage site in the second intracellular loop.

FIG. 15 is a schematic showing the DNA (SEQ ID NO:9) and amino acid (SEQ ID NO:7) sequences of a β2-adrenergic receptor modified to contain a TEV protease cleavage site in the third intracellular loop.

FIG. 16 is a schematic showing the amino acid sequence of μ-opioid receptor (SEQ ID NO:12) and modifications that can be made within the second intracellular loop (SEQ ID NO:14) or within the third intracellular loop (SEQ ID NO:16) to insert a protease cleavage site (exemplified by tobacco etch virus (TEV)) that can serve as a conformationally sensitive probe for ligand binding.

FIG. 17 is a schematic showing the DNA (SEQ ID NO:11) and amino acid (SEQ ID NO:12) sequences of a μ (mu) opioid receptor.

FIG. 18 is a schematic showing the DNA (SEQ ID NO:13) and amino acid (SEQ ID NO:14) sequences of a μ opioid receptor modified to contain a TEV protease cleavage site in the second intracellular loop.

FIG. 19 is a schematic showing the DNA (SEQ ID NO:15) and amino acid (SEQ ID NO:16) sequences of a p opioid receptor modified to contain a TEV protease cleavage site in the third intracellular loop.

FIG. 20 is a schematic illustrating various “membrane spanning motifs” of MSST proteins. Membrane spanning motifs minimally composed of extracellular region(s), transmembrane region(s), and intracellular region(s) present in MSST proteins. In general, generic MSST proteins comprises one or more such membrane spanning motifs. Binding of a drug (agonist or antagonist) to, for example, the extracellular domains or transmembrane domains results in movement of the transmembrane domains that can be detected by a conformationally sensitive, detectable probe on one of the intracellular domains, either the sequences connecting the transmembrane domains or the carboxyl terminal domain.

FIG. 21 is a schematic illustrating generic structures of exemplary MSST proteins. The generic structure of a GPCR, a potassium ion channel, and a transporter protein are exemplified.

### DETAILED DESCRIPTION OF INVENTION

Before the present compositions, assays and methods are described, it is to be understood that this invention is not limited to particular protocols and/or embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded-limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a GPCR” includes a plurality of such GPCRs and reference to “the ligand” includes reference to one or more ligand and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

“Membrane-spanning, signal-transducing protein” (also referred to herein as an “MSST protein”) refers to a protein having at least one transmembrane domain, at least one extracellular domain, and at least one intracellular domain. Where the MSST protein comprises two or more transmembrane domains, the transmembrane domains are linked by at least one intracellular loop or at least one extracellular loop. Exemplary MSST proteins include, but are not necessarily limited to, GPCRs, ion channels, and transporter proteins.

“Intracellular loop” and “extracellular loop” refer to amino acid sequences connecting adjacent transmembrane domains of a membrane spanning protein which, when present in their native configuration in a cell, are located on the cytoplasmic side and the extracellular side of the cellular membrane, respectively. Use of these terms herein is not meant to be limiting to the position of these loops within cells, but rather is only used for clarity and convenience to refer to the relative position of these domains within the membrane spanning protein relative to a membrane in which the protein is positioned. That is, an intracellular loop is positioned on a side of the membrane that is opposite from that of an extracellular loop.

“Transmembrane region” or “transmembrane domain” refers to a portion of a protein that resides primarily in a membrane.

“Conformationally sensitive region” of an MSST protein refers to a portion of the MSST protein that exhibits distinct conformational changes in the presence of a ligand compared to the absence of a ligand of the MSST protein, and thus are suitable for use or modification or use as conformationally sensitive detectable probes. Exemplary conformationally sensitive regions of interest include intracellular loops, extracellular loops, N-terminal regions, and C-terminal regions.

The term “conformationally sensitive detectable probe” as used herein refers to a moiety on a naturally occurring or modified MSST protein that provides a change in a detectable signal upon interaction of the protein with a ligand, particularly with ligands having either agonist activity (e.g., activity as a full or partial agonist) or inverse agonist activity. One exemplary conformationally sensitive detectable probe is a detectable chemical label (e.g., a fluorescent moiety) that is attached to an amino acid residue at a conformationally sensitive site (e.g., within the third intracellular loop of a GPCR (e.g., an amino acid residue corresponding to Cys265 of β2-AR)), so that interaction of the MSST protein with an agonist-results in a change in the detectable signal of the detectable chemical label (e.g., a decrease in signal due to agonist binding).

Another exemplary conformationally sensitive detectable probe is an integral detectable moiety of the MSST protein, which moiety can comprise, for example, an amino acid sequence defining, for example, a protease cleavage site or an immunodetectable epitope. The integral moiety by be naturally occurring or introduced using recombinant techniques.). An integral detectable moiety is usually positioned in a hydrophilic sequence adjacent to a transmembrane that undergoes a conformational change following ligand binding (e.g. the third loop of the GPCR), so that the protease cleavage site becomes more or less accessible following interaction with a ligand.

“Detectable chemical label” as used herein refers to any suitable detectable label which can be attached to or introduced into a conformationally sensitive region of an MSST protein, and which provides a distinguishable detectable signal(s) according to the conformational state of the protein (e.g., the conformation of the protein in the presence versus the absence of ligand).

“Integral detectable moiety” and “detectable integral moiety” are used interchangeably herein to refer to an amino acid sequence within a conformationally sensitive region of a MSST protein, which sequence differs in its accessibility to a recognition partner according to the conformational state of the protein (e.g., the conformation of the protein in the presence versus the absence of ligand). Exemplary integral detectable moieties include a protease cleavage site (which has a site-specific protease as its recognition partner) and an immunodetectable epitope (which has as its recognition partner an antibody that specifically binds the epitope). Detectable integral moieties can be endogenous to the MSST protein or introduced (e.g., through recombinant techniques and thus are “heterologous” to the MSST protein (i.e., an amino acid sequence that is of an origin different than that of the MSST protein being modified). In a preferred embodiment, the integral detectable moiety is introduced.

The terms “epitope tagged protein” and the like are used interchangeably herein to mean an artificially constructed proteins having one or more heterologous epitope domain(s).

The term “biological system” as used herein refers to any system in which the molecular responses to the activation of G proteins, e.g., activation through GPCRs, can be measured. The biological systems may be in vitro (e.g., membrane preparations or cell culture).

By “immobilization phase” is meant a support to which an MSST protein or membrane preparation comprising an MSST protein can be reversibly or irreversibly stably attached, usually irreversibly stably attached. By “stably attached” is meant stably associated is meant that the MSST protein maintains its position relative to the support under assay conditions. The immobilization phase can be of any suitable form including solid, semi-solid, and the like. Usually, the immobilization phase comprises the well of an assay plate but the invention is by no means limited to this embodiment. For example, the immobilization phase can comprise a discontinuous immobilization phase of discrete particles, or it may comprise a flat surface. The immobilization phase can be formed from a number of different materials, e.g., polysaccharides (e.g. agarose), polyacrylamides, polystyrene, polyvinyl alcohol, silicones and glasses. The surface of the immobilization phase can be modified to allow for specific and/or oriented interaction of the receptor with the surface.

By “membrane” is meant a natural membrane (e.g., plasma membrane or fragment from a eukaryotic cell (e.g., insect)), an artificial membrane, or a surrogate membrane (e.g., detergent micelle).

By “well” is meant a recess or holding space in which an aqueous sample can be placed. The well is provided in an “assay plate” which is formed from a material (e.g. polystyrene) that optimizes adherence of cells (having the receptor or receptor construct) or membrane preparations thereto. The individual wells of the assay plate can have any suitable shape, including but not limited to a round bottom well and a flat bottom well. In a particular embodiment of the invention, the assay plate comprises between about 30 to 200 individual wells, usually 96 wells, and is designed to allow for automation of the assay.

By “array” as used in the context of “MSST protein array” is meant a distribution of MSST proteins so that MSST proteins (or pools of MSST proteins) are provided at spatially-addressable coordinates, usually at defined X-Y coordinates, so as to assess interactions of the MSST proteins (or pooled MSST proteins) with other molecules, e.g., such that detectable signal from a given coordinate on the array can be matched to the MSST protein (or pool of MSST proteins) at that coordinate.

The term “ligand” as used herein refers to a naturally occurring or synthetic compound that binds to a protein receptor. Upon binding to a receptor, ligands generally lead to the modulation of activity of the receptor. The term is intended to encompass naturally occurring compounds, synthetic compounds and/or recombinantly produced compounds. As used herein, this term can encompass agonists, antagonists, and inverse agonists.

The term “agonist” as used herein refers to a molecule or substance that binds to or otherwise interacts with a receptor or enzyme to increase activity of that receptor or enzyme. Agonist as used herein encompasses both full agonists and partial agonists.

The term “antagonist” as used herein refers to a molecule that binds to or otherwise interacts with a receptor to block (e.g., inhibit) the activation of that receptor or enzyme by an agonist.

The term “inverse agonist” as used herein refers to a molecule that binds to or otherwise interacts with a receptor to inhibit the basal activation of that receptor or enzyme.

The term “receptor” as used herein refers to a protein normally found on the surface of a cell which, when activated, leads to a signaling cascade in a cell.

The term “functional interaction” as used herein refers to an interaction between a receptor and ligand that results in modulation of a cellular response. These may include changes in membrane potential, secretion, action potential generation, activation of enzymatic pathways and long term structural changes in cellular architecture or function.

The terms “G protein coupled receptors” and “GPCRs” as used interchangeably herein include all subtypes of the opioid, muscarinic, dopamine, adrenergic, adenosine, rhodopsin, angiotensin, serotonin, thyrotropin, gonadotropin, substance-K, substance-P and substance-R receptors, melanocortin, metabotropic glutamate, or any other GPCR known to couple via G proteins. This term also includes orphan receptors that are known to couple to G proteins, but for which no specific ligand is known.

The term “G protein subunit” as used herein can refer to any of the three subunits, α, β or γ, that form the heterotrimeric G protein. The term also refers to a subunit of any class of G protein, e.g., Gs, Gi/Go, Gq and Gz. In addition, recitation of a specific subunit (e.g., Gα) is intended to encompass that subunit in each of the different classes, unless the class of G protein is specifically otherwise specified.

“Ion channel” as used herein refers to a protein crossing the lipid bilayer of a cell, which, in a regulated manner, transports solutes and/or water across cell membranes. Channels are responsible for generating and propagating electrical impulses in excitable tissues in the brain, heart, and muscle, and for setting the membrane potential of excitable and non-excitable cells. Exemplary ion channels include sodium channels, potassium channels, and calcium channels, as well as ligand gated ion channels such as serotonin, glutamate, and γ-aminobutyric acid (GABA) channels.

“Transporter protein” as used herein refers to specific high-affinity neurotransmitter transporters located in the plasma membranes of cells. These proteins function to move their substrate from one side of a membrane to the other side in a regulated manner. This designation includes members of the following sub-families gamma (γ)-aminobutyric acid transporters, monoamine transporters, amino acid transporters, bacterial transporters, and “orphan” transporters.

The abbreviations used herein include:

GPCR for G protein-coupled receptor;

β2 AR (or b2AR or beta2AR) for β2 adrenoceptor;

FM for fluorescein maleimide;

Gα, for an α subunit of a G-protein

Gₛα, for an α subunit of the stimulatory G-protein;

AC for adenylyl cyclase;

(³H)DHA for (³H)dihydroalprenol;

GTPγS for guanosine 5′-O-(3-thiotriphosphate);

ISO for (−)isoproterenol;

DOB for dobutamine;

ALP for (−) alprenolol; and

ICI for ICI-118,551.

### Overview

The present invention is based on the discovery that conformationally sensitive probes can be used to detect interactions between a MSST protein (such as a GPCR, a protein channel, a transporter protein, and the like) and ligands by direct detection of ligand-induced conformational changes in the protein.

Monitoring of ligand-induced conformational change according to the invention is accomplished by modifying a MSST protein with a conformationally sensitive probe at a specific, conformationally sensitive site on the protein. Conformationally sensitive sites useful in the invention are generally regions of the MSST protein other than the transmembrane domain, and which extend past a membrane in which the MSST protein is present. Examples include intracellular loops, extracellular loops, and C-terminal regions of an MSST protein. Conformationally sensitive, detectable probes useful in the invention are of generally two classes. The first class comprises chemical detectable labels, which can be attached to endogenous or modified amino acid residues present in a conformationally sensitive region of a MSST protein. Examples of detectable chemical labels include fluorophores, electron paramagnetic resonance (EPR) labels, and nuclear magnetic resonance (NMR) labels. When detectable chemical labels are used as conformationally sensitive probes, receptor-ligand interactions can be monitored using, for example, a fluorescence-based assay. In the case where MSST protein is labeled directly with the fluorescent probe, the interaction assay can be performed with purified, detergent solubilized MSST protein.

A second class of conformationally sensitive detectable probes are integral detectable moieties present on the MSST protein. Such integral detectable moieties are defined by amino acid sequences present in the MSST protein which differ in their accessibility to a recognition partner according to conformational changes in the MSST protein that are associated with the presence and absence of ligand. Exemplary integral detectable moieties include, but are not necessarily limited to, protease cleavage sites and immunodetectable epitopes. In this embodiment, the assay can be performed on purified MSST protein or with a MSST protein-enriched membrane fragment.

In each embodiment of the invention, modulation of MSST protein activity is detected by detecting a change in detectable signal elicited by the conformationally sensitive detectable probe, e.g., by detection of a change (increase or decrease) in signal from a chemical label, by detection of an increase or decrease in protease cleavage products, an increase or decrease in antibody binding to an immunodetectable epitope. The increase or decrease in detectable signal can be relative to a control level of detectable signal, where the control can be a level of detectable signal in the absence of the candidate agent (e.g., negative control), in the presence of a known MSST protein modulator (e.g., positive control, e.g., agonist or antagonist), and the like. For example, the detectable signal of the conformationally sensitive probe of a MSST protein is compared in the presence or absence of candidate agent (or drug or known ligand), where a statistically significant difference in signal is indicative of MSST protein modulation. Generally, a decrease or increase in signal relative to a control level of signal of at least about 10%, usually at least about 20%, more usually at least about 50% to 100% or more is indicative of modulation of MSST protein activity.

All embodiments of the invention allow the generation of arrays consisting of different MSST proteins such that MSST protein-ligand interactions can be assessed in multiple proteins simultaneously.

Each of the elements of the invention will now be described in more detail.

Membrane-Spanning, Signal-Transducing Protein

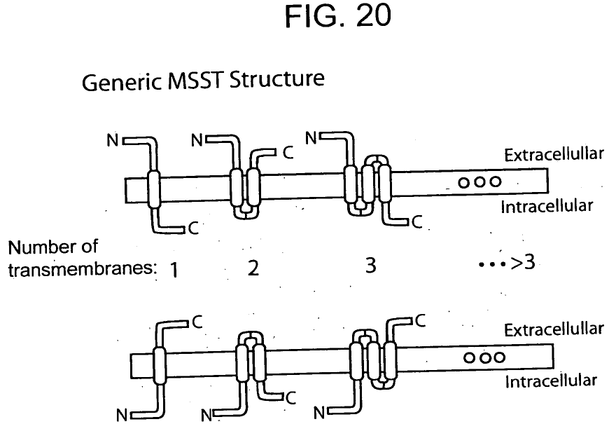

Membrane-spanning, signal-transducing proteins (“MSST” proteins) (also referred to herein as an “MSST protein”) is defined herein as a protein having at least one membrane spanning motif, which motif minimally comprises at least one transmembrane domain, at least one extracellular domain, and at least one intracellular domain. Where the MSST protein comprises two or more transmembrane domains, the transmembrane domains are linked by at least one intracellular or one extracellular loop, .e.g., where the MSST protein comprises two or more membrane spanning motifs, the C-terminus of a first motif is joined to the N-terminus of a second motif (i.e., the transmembrane domains are joined by alternating intracellular and extracellular domains). FIG. 20 provides a schematic of exemplary MSST protein structures, with varying numbers of membrane spanning motifs (and thus varying numbers of transmembrane domains). In general, as illustrated in FIG. 20, “n” represents the number of membrane-spanning motifs, where n in typical MSST proteins ranges from 1 to 12 or more, and is usually greater than or equal to 2. For example, in the context of the GPCR protein, “n” is usually 7.

Conformationally sensitive regions of MSST proteins suitable for use as, or modification to have, a conformationally sensitive probe are generally regions of the MSST protein that are accessible to the appropriate detection method (e.g., a region that is susceptible to detection using a conformationally sensitive probe), such that the accessibility of the region changes with changes in the conformation of the adjacent transmembrane domains of the MSST protein that result from ligand interaction.

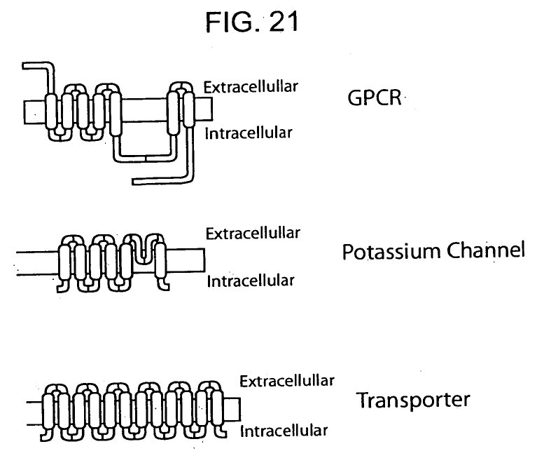

FIG. 21 is a schematic illustrating structures of exemplary MSST proteins. The generic structure of a GPCR, a potassium ion channel, and a transporter protein are exemplified. Each of these exemplary MSST proteins contain conformationally sensitive regions suitable for adaptation as conformationally sensitive detectable probes.

As noted above, exemplary MSST proteins include, but are not necessarily limited to, GPCRs, ion channels, and transporter proteins. Each of these classes of proteins are discussed in more detail below.

GPCRs

Exemplary GPCRs that can be used in the screening assays of the invention include, but are not necessarily limited adrenoceptors, opioid receptors, and the like. Further exemplary GPCRs that can be used in the present invention are listed in the table below. The GPCRs are classified according to the type of ligand they naturally bind.

Table of Exemplary GPCRs

| Peptide ligands | | Other Receptors |
|---|---|---|
| Angiotensin receptors | Releasing hormone | |
| | receptors | |
| | (LHRH, GHRH) | |
| Bombesin receptors | Somatostatin receptors | |
| Bradykinin receptors | Tachykinin receptors | |
| Calcitonin, parathyroid | Thrombin/protease | |
| hormone, secretin | receptors | |
| receptors | | |
| Chemokine receptors | Vasopressin/oxytocin | |
| | receptors | |
| Chemotactic peptide | Glycoprotein hormones | Odorant/olfactory |
| receptors (fMLP) | receptors (TSH, FSH, | and gustatory |
| | LH) | receptors |
| C5A receptor | Melanocortins receptors | Opsins |
| Cholecystokinin/gastrin | Neuropeptide Y receptors | Viral receptors |
| receptors | | |
| Corticotropin (ACTH) | Neurotensin receptors | Orphan receptors |
| receptor | | |
| Endothelin receptors | Opioid peptides | |
| | receptors (mu, delta, | |
| | kappa & opioid | |
| | like) | |

Natural small molecule ligands

| | | |
|---|---|---|
| Acetylcholine | Dopamine receptors | Prostanoids and |
| (muscarinic) | | PAF |
| receptors | | receptors |
| Adenosine and adenine | Histamine receptors | Serotonin receptors |
| nucleotide receptors | | |
| Adrenergic receptors | Cannabinoids | Metabotropic |
| | receptors | glutamate and |
| | | calcium receptors |

The GPCRs that are involved in known biological responses (e.g., responses to hormones and neurotransmitters, as well as odorants) and orphan GPCRs can be studied using assays and apparatus of the invention. An assay using an array of membranes or proteins, each sample of the array having a particular GPCR of interest, can be exposed to the stimulus (e.g., natural or synthetic ligand, e.g., candidate drug), and the activity of each sample of the array can be determined. This can identify ligands for multiple receptors in a high-throughput manner.

The high-throughput assays of the invention can be especially useful in determining the spectrum of GPCRs, that are activated or inverse agonized by a specific substance or mixture of substances. For example, a solution containing one or more compounds can be contacted with an array of membrane preparations each having a particular GPCR of interest, and the GPCRs activated or suppressed can be identified by detection of a conformational change in the GPCR. This can classify the compound(s) as active at one or more specific GPCRs.

In another example, an assay using the apparatus of the invention can be used to identify the ligands that bind to and modulate GPCRs of unknown activity, e.g., orphan receptors. Identification of ligands that modulate specific receptors can lead to a better understanding of the functional role of that particular receptor.

The invention can also be used to characterize the composition of solution. For example, an array of odorant receptors can be used to define the composition of specific odorants in perfume.

The invention can also be used to identify proteins that interact with a GPCR, such as proteins that regulate the function of the GPCR or proteins that are regulated by the GPCR.

Other uses are also envisioned, as will be apparent to one skilled in the art upon reading the present disclosure.

Conformationally Sensitive Regions of GPCRs

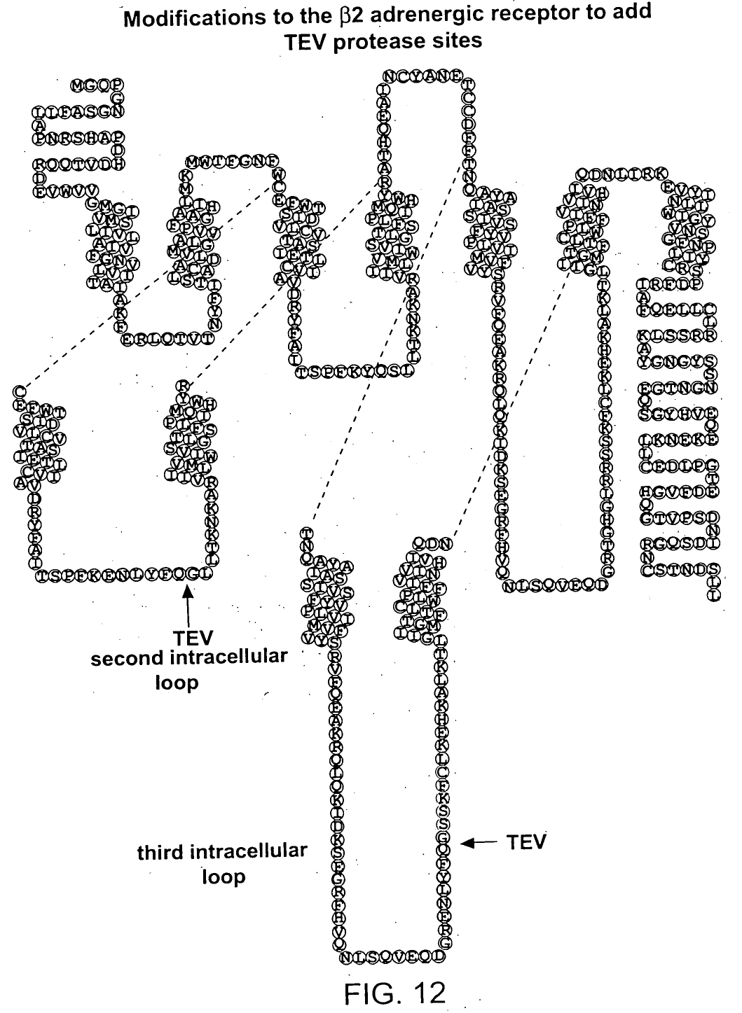

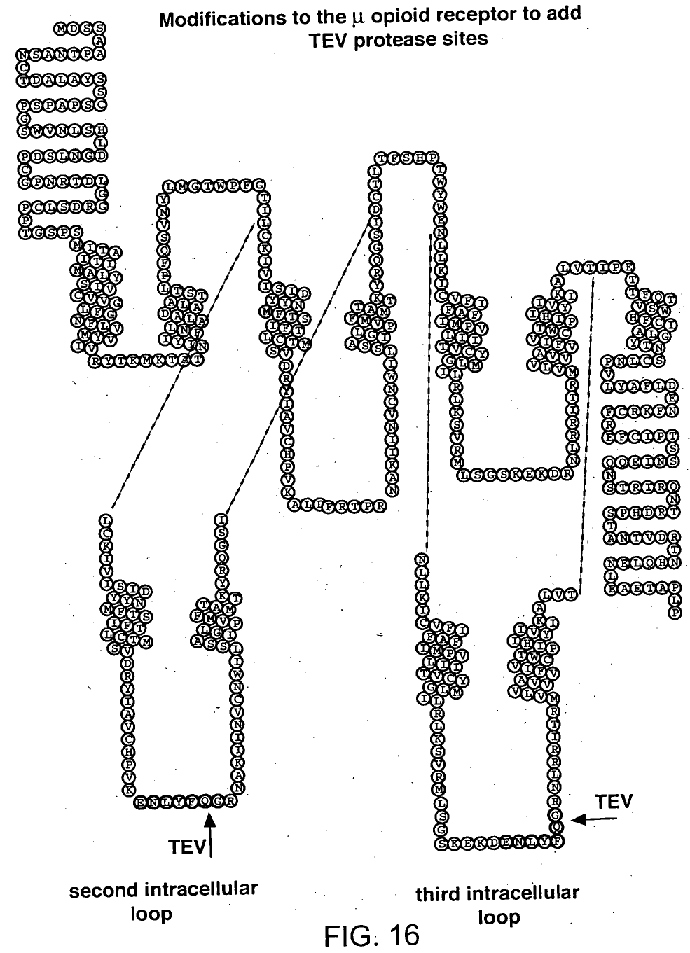

GPCRs contain several regions that-are conformationally sensitive and are suitable for adaptation to include a conformationally sensitive detectable probe. In general, such conformationally sensitive regions are located within an N-terminal domain (i.e., a portion of the N-terminal end of the protein that is located primarily outside of a membrane), a C-terminal domain (i.e., a portion of the C-terminal end of the protein that is located primarily outside of a membrane), an intracellular loop, and/or an extracellular loop. In one embodiment, the amino acid residue(s) modified to contain or provide a conformationally sensitive detectable probe are those residues corresponding to: 1) the third intracellular loop present in GPCR proteins; 2) the second intracellular loop present in GPCR proteins; 3) the carboxyl terminus present in GPCR proteins; and/or 4) the amino terminus present in GPCR proteins. These structural regions are conserved in GPCRs. Modified GPCRs include those modified to contain a conformationally sensitive detectable probe in one or more of these regions. Examples of modifications of two exemplary GPCRs, the β₂-AR and the μ opioid receptor, are illustrated in the Examples below and in FIGS. 12 and 16.

Ion Channels

Exemplary ion channels that can be used in the screening assays of the invention include, but are not necessarily limited to voltage-gated potassium, sodium, and calcium channels, and cation channels gated by intracellular cyclic nucleotides or ATP. In addition, there are a variety of neurotransmitter-specific ligand-gated channels having distinct ligand-binding, ion selectivity, and conductance properties. The acetylcholine-, serotonin-, or glutamate-gated channels, at excitatory synapses, create an environment that allows the passage of cations, whereas the glycine and γ-aminobutyric acid-gated ion channels, at inhibitory synapses, create the same for anions. The glutamate-gated channels are further subdivided according to their selective agonists as the γ-amino-3-hydroxy-5-methyl-4-isoxazole proprionic acid (AMPA), kainate, and N-methyl-D-aspartate (NMDA) receptors. The AMPA and kainate receptors conduct mainly monovalent cations, while the NMDA receptor has a slower response and is permeable to Ca in a voltage-dependent and Mg dependent manner.

The ion channels that are involved in biological responses (e.g., neurotransmission, etc.) can be determined using assays and apparatus of the invention. An assay using an array of membranes or proteins, each sample of the array having a particular ion channel of interest, can be exposed to the stimulus, and the activity of each sample of the array can be determined. This can identify ligands for multiple ion channels in a high-throughput manner.

The high-throughput assays of the invention can be especially useful in determining the spectrum of ion channels, e.g., NMDA receptors, that are activated or inverse agonized by a specific substance or mixture of substances. For example, a solution containing one or more compounds can be contacted with an array of membrane preparations each having a particular ion channel of interest, and the ion channels activated or suppressed can be identified by detection of a conformational change in the ion channel. This can classify the compound as important in modulating the function of one or more specific ion channels.

In another example, an assay using the apparatus of the invention can be used to identify the ligands that bind to and modulate ion channels of unknown activity, e.g., orphan ion channels. Identification of ligands that modulate specific-ion channels can lead to a better understanding of the functional role of that particular ion channel.

The invention can also be used to identify proteins that interact with an ion channel, such as proteins that regulate the function of the ion channel or proteins that are regulated by the ion channel.

Other uses are also envisioned, as will be apparent to one skilled in the art upon reading the present disclosure.

Conformationally Sensitive Regions of Ion Channels

Ion channels contain several regions that are conformationally sensitive and are suitable for adaptation to include a conformationally sensitive detectable probe. In one embodiment, the amino acid residue(s) modified to contain or provide a conformationally sensitive detectable probe are those residues corresponding to amino acid residues within: 1) the pore loop (SS1-SS2 or H5 loop) that connects transmembrane segments five and six on each channel domain; 2) portions of either the “hinged lid” or “ball and chain” regions that function to inactivate the pore through which ions travel; 3) loops linking portions of the “transducer box”, which consists of a region joining the transmembrane and cytoplasmic domains of the ion channel, 4) loop regions connected to the fourth transmembrane domain (S4), which is responsible for detecting voltage changes, 5) the charged loop between the amino terminal tetramerization domain (T1) and the first transmembrane domain (S1), 6) portions connecting the hinged S1S2 ligand binding domains, 7) the loop that serves as a loose lid over the binding site cavity, 8) the neurotransmitter binding site. These structural regions are conserved in ion channel subfamilies. Modified ion channels include those modified to contain a conformationally sensitive detectable probe in one or more of these regions (see, e.g., Herbert, S. C., Am. J. Med. 104:87-98, Choe, S., Nat. Neurosci. 3:115-121, Madden, D. R., Nat. Neurosci. 3:91-101, Karlin, A., Nat. Neurosci., 3:102-114, Yi et al., Proc. Natl. Acad. Sci. 98:11016-11023, Mendieta et al., Proteins 44:460-469, Abele et al., J. Biol. Chem. 275:21355-21363, Dani et al., Curr. Opin. Neurobiol. 5:310-317, Hanlon et al., Biochem. 41:2886-2894, Unwin, N. Cell 72 (Suppl): 31-41, Karlin et al., Neuron 15:1231-1244, MacKinnon, R. Neuron 14:889-892, Catterall, W. A. Annu. Rev. Cell Dev. Biol. 16-521-55, Dingledine et al. Pharmacol. Rev. 51:7-61).

Transporter Proteins

Exemplary transporters that can be used in the screening assays of the invention include, but are not necessarily limited to transporters for the substrates betaine, creatine, dopamine, γ-aminobutyric acid, glycine, noradrenaline, serotonin, proline, and taurine, and the like. Transporters are classified according to the type of substrate they naturally bind.

Transporters that are involved in biological responses (e.g., neurotransmitter reuptake, etc.) can be determined using assays and apparatus of the invention. An assay using an array of membranes or proteins, each sample of the array having a particular ion channel of interest, can be exposed to the stimulus, and the activity of each sample of the array can be determined. This can identify ligands for multiple transporters in a high-throughput manner.

The high-throughput assays of the invention can be especially useful in determining the spectrum of transporters, e.g., serotonin transporters, that are activated or inverse agonized by a specific substance or mixture of substances. For example, a solution containing one or more compounds can be contacted with an array of membrane preparations each having a particular transporter of interest, and the transporter activated or suppressed can be identified by detection of a conformational change in the transporter. This can classify the compound as important in modulating the function of one or more specific transporter.

In another example, an assay using the apparatus of the invention can be used to identify the ligands that bind to and modulate transporters of unknown activity, e.g., orphan transporters. Identification of ligands that modulate specific transporters can lead to a better understanding of the functional role of that particular transporter.

The invention can also be used to identify proteins that interact with a transporter such as proteins that regulate the function of the transporter or proteins that are regulated by the transporter.

Other uses are also envisioned, as will be apparent to one skilled in the art upon reading the present disclosure.

Conformationally Sensitive Regions of Transporters

In one embodiment, the amino acid residue(s) modified to contain or provide a conformationally sensitive detectable probe are those residues corresponding to: 1) the first extracellular loop, 2) the first intracellular loop, 3) the third intracellular loop, 4) and the second extracellular loop, with or without transmembrane residues located at the extracellular surface of the seventh transmembrane and the eighth transmembranes (see, e.g., Ferrer et al., Proc. Natl. Acad. Sci USA 95:9238-9243, Loland et al., J. Biol. Chem. 274: 36928-36934, Lopez-Cocuera et al., J. Biol. Chem. 276: 43463-43470, Androutsellis-Theotokis et al., J. Biol. Chem. 276:45933-45938, Ni et al., J. Biol. Chem. 276:30942-30947, and MacAulay et al., J. Biol. Chem. 276:40476-40485). These structural regions are conserved in transporters. Modified transporters include those modified to contain a conformationally sensitive detectable probe in one or more of these regions

Assays of the Present Invention

The methods of the invention for detecting or identifying MSST protein activation are important for numerous applications in medicine and biology. The present invention provides methods including: (1) methods for rapidly and reproducibly screening for new drugs affecting selected MSST proteins, (2) methods for identifying native ligand(s) for MSST proteins (such as orphan GPCRs), (3) methods for detecting the presence of a ligand of a MSST protein in a sample, and (4) methods for identifying other components of the signaling cascade. The basic assays described herein and variations thereof can also be used in other applications, as will be apparent to those skilled in the art upon reading the present application.

A significant advantage of the assays of the invention is that they can directly detect interaction of a molecule (compound, peptide, or protein) with a MSST protein either qualitatively or quantitatively, and thus are particularly amenable to high-throughput screening of large numbers of MSST-proteins. For example, the assay can be conducted using two or more different MSST proteins, where different proteins can be different due to differences in naturally-occurring or artificially-introduced amino acids sequences (e.g., a native and mutated version of a βAR, a native βAR and a native opioid receptor, a modified GPCR having different conformationally sensitive detectable probes and/or having different probes at different conformationally sensitive sites in the protein, etc.).

The assay can be conducted using a plurality of different MSST proteins (e.g., three or more, five or more, ten or more, 20 or more, 50, 100, 200, 250, 400, or 500 or more, and the like). The different MSST proteins can be provided in membranes or micelles, or can be provided in the membrane or micelle, where induction of activity of the MSST protein can be detected using different detectable labels. Detection of activity of compounds on different MSST proteins can be accomplished by differential labeling of the proteins (e.g., particularly where two or more MSST proteins are provided in the same membrane). In general, a plurality of MSST proteins can be screened by distinguishing the different proteins based on their location on an array (e.g., each MSST protein is positioned on an immobilization phase at a known coordinate, so that detection of a change in detectable label at that coordinate (e.g.; detection of a change in fluorescent signal at that coordinate) can be associated with activity of the compound on the MSST protein at that same coordinate). In another embodiment, the different MSST proteins can be screened in pools. Pools of interest for further screening can then be divided and subdivided to further determine which MSST protein(s) in the pool have activity modulated by the candidate agent.

The MSST proteins screened can represent a diverse collection of MSST proteins, or can represent a collection of MSST proteins having a role in a biological phenomenon of interest. This can be useful, for example, in determining the receptors activated by a particular drug, or receptors that are activated upon exposure to a particular stimulus, so as to modulate activity of a MSST protein in a biological responses (e.g., responses to hormones and neurotransmitters, as well as odorants).

Production of MSST proteins (for modification and labeling) can be accomplished using any suitable host cell (e.g., mammalian, yeast, insect, or bacterial). In one embodiment of particular interest, the host cells are insect cells. Methods for expression of recombinant MSST proteins, as well as methods for isolation of such recombinant MSST-proteins and methods of production of membranes containing MSST proteins, are well known in the art (see, e.g., Kobilka Anal. Biochem. 231(1):269-71 (1995); Gether et al. J. Biol. Chem. 270(47):28268-75 (1995)).

Candidate Agents

Identification of compounds that modulate MSST proteins activity can be accomplished using any of a variety of drug screening techniques as described in more detail below. Of particular interest is the identification of agents that have activity in affecting MSST proteins function. Such agents are candidates for development of treatments for conditions associated at least in part with MSST proteins activity. Of particular interest are screening assays for agents that have a low toxicity for human cells. The term “agent” as used herein describes any molecule, e.g. protein or pharmaceutical, with the capability of altering (i.e., eliciting or inhibiting) activity of a MSST protein. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, usually at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting MSST protein activity s) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Screening Assays

In general, the assays of the invention involve detection of a conformational change of a MSST protein through detection of a conformationally sensitive probe.

In one embodiment, the conformationally sensitive probe is a detectable chemical label, e.g., bound to a residue within a conformationally sensitive region (e.g., a third intracellular loop of a GPCR). In another embodiment, the conformationally sensitive probe is a detectable integral moiety (such as a protease cleavage site), where the accessibility of the site to interaction with its recognition partner (e.g., a protease) changes depending upon the conformation of the MSST protein (e.g., the conformation of the MSST protein in the presence or absence of ligand).

Each of these embodiments will now be described in more detail.

MSST Proteins Suitable for use in Screening Assays

As noted above, the MSST proteins useful in screening assays according to the invention contain or are modified to contain a conformationally sensitive, detectable probe, which probe can be a chemical label or a detectable integral moiety. Exemplary embodiments are described in more detail below.

MSST Proteins Adapted to Comprise a Detectable Chemical Label.

In one embodiment, the conformationally sensitive detectable probe is a detectable chemical label that is attached to at least one amino acid residue of a MSST protein in a conformationally sensitive structural domain of the MSST protein, e.g., an amino acid residue of the third intracellular loop of a GPCR.

Various detectable chemical labels include radioisotopes, fluorophores, chemiluminescers, nitroxide spin labels or other label that provides a change in detectable signal upon a change in conformation of the MSST protein. Detectable chemical labels of the invention also include those for use in FRET (fluorescence resonance energy transfer) and BRET detection systems., which systems are well known in the art. Fluorescent labels are of particular interest as detectable chemical labels.

An isolated MSST protein having a detectable chemical label can be assayed in detergent solution or fixed to a substrate such as a glass slide or an immobilized membrane (e.g., lipid bilayer, micelles, inside-out vesicles, and the like). Interaction of a ligand with the chemically labeled MSST protein causes a conformational change in the protein, which in turn changes the detectable signal (e.g., increase or decrease in the signal relative to a control) from the detectable chemical label. Ligand-induced changes in intensity of the detectable chemical label can be studied using conventional methods, e.g., fluorimeters or array readers. The change in detectable signal upon interaction of the detectably, chemically labeled MSST protein with a ligand can be used to, for example, assess the affinity of the ligand for the receptor. In addition or alternatively, where the MSST proteins are provided on an array (or the ligands are provided on an array), the change in detectable signal at a location(s) on the array, as well as the relative amount of change in the detectable signal, can be used to identify protein-ligand interactions, and provide for identification of the corresponding MSST protein (or ligand) on the array by virtue of the assigned array coordinates.

Modifications to Modulate Assay Output of Chemically Labeled MSST Proteins

In some embodiments, the assay can be modified to enhance detection of ligand-MSST protein binding. For example, in some embodiments, the detectable signal will not change upon ligand binding to the MSST protein. However, the addition of reagents (e.g., fluorescence quenchers) that partition into specific environments around the receptor (e.g., within the aqueous environment or within the lipid bilayer) can be used to reveal conformational changes that occur upon receptor-ligand interactions. Exemplary fluorescent quenching agents include, but are not necessarily limited to, the nitroxide labeled fatty acid CAT-16, 5-doxyl stearate (5-DOX), potassium iodide (KI), and the like. In this embodiment, induction of a conformational change in the MSST protein upon ligand binding results in movement of the detectable label (e.g., fluorophore) toward or away from a quenching reagent, thus modifying the detectable signal.

For example, where the detectable label is a fluorescent label, the detectable signal can be enhanced by adding a quenching agent to the detergent micelle or to the lipid bilayer. For example, CAT-16 is a modified fatty acid that has a nitroxide spin label covalently attached to the polar head group. Studies on β2-AR labeled with fluorescein at Cys265 show that agonist-induced changes in fluorescence are enhanced in the presence of CAT-16, suggesting that agonist-induced structural changes lead to the movement of fluorescein on Cys265 closer to the polar surface of the detergent micelle. For some receptors, it may be necessary to modify one or more labeling site(s) for the fluorophore to obtain an optimal signal. Thus, modified receptors having reactive cysteines at positions −2, −1, +1 and +2 relative to the position homologous to Cys265 in the β2-AR can be generated

To improve the signal to noise, a second detectable chemical label (e.g., a second fluorescent label having a different excitation and emission spectrum) can be added to a conformationally insensitive domain on the receptor. The detectable signal of the second detectable chemical label would be used to control for variations in signal intensity due to differences in the amount of receptor protein. The signal would therefore be, for example, the ratio of conformationally sensitive probe (Ps) to the conformationally insensitive probe (Pi). The intensity of Ps will change when the receptor is bound to agonists and partial agonists, but will not change when the receptor is bound to antagonists. Antagonist binding can, however, be detected by stabilization of receptor against denaturation by reducing agents.

Modification of MSST Proteins to Provide for Detectable Chemical Label

MSST proteins can be modified to comprise one or more amino acid residues within a conformationally sensitive domain that are suitable for attachment to a detectable chemical label. For example, where a GPCR to be analyzed does not have an amino acid residue analogous to the cysteine residue at position 265 of β2-AR, the GPCR can be modified using available recombinant techniques to introduce such a cysteine residue (e.g., using site-specific mutagenesis or other available techniques). Alternatively, the GPCR to be analyzed can have an intracellular loop analogous to the third intracellular loop of β2-AR replaced with the third intracellular loop of the β2-AR.

MSST proteins of interest can be modified using standard recombinant DNA technology to include an epitope tag at the amino terminal end, carboxyl terminal end, or both. For example, a MSST protein can be modified to have an amino terminal Flag epitope and a carboxyl terminal hexahistidine sequence. These modifications facilitate purification of the protein. In addition, the intracellular domains of the MSST proteins can be modified so that all native cysteines, other than the consensus palmitoylation sites, are mutated to serine or alanine to facilitate use of a detectable chemical label.

The MSST proteins can be modified to incorporate amino acids that are susceptible to specific modification using a detectable chemical label. Cysteine residues are of particular interest for introduction, substitution, addition, or as a replacement residue for a native amino acid residue of a MSST protein. For example for a GPCR, a cysteine can be added to the cytoplasmic end of TM6 corresponding to Cys265 in the human β2-AR. This can also be accomplished by an exchange of the entire third intracellular loop of the GPCR for the third intracellular loop of the β2AR. The modified MSST proteins can be expressed in insect cells or other host cells using standard recombinant methods.

After sufficient time for protein production, cells are harvested and intact cells are treated with iodoacetamide to block native cysteines in the extracellular domains of the MSST protein. This will prevent nonspecific labeling of these sites with the fluorescent label. Cells are then lysed, and membranes prepared. The membranes can be frozen for years (e.g. at −80° C.). Receptors can be purified by chromatography on Flag affinity resin where the Flag epitope is used. The purified receptor is then labeled with fluorescein (or another environmentally sensitive fluorophore) and the unreacted fluorophore is separated from the labeled protein using Ni chelating chromatography.

MSST Proteins Adapted to Comprise a Detectable Integral Moiety.

In one embodiment, the conformationally sensitive detectable probe is a detectable integral moiety, which moiety comprises an amino acid sequence within the amino acid sequence of an MSST protein. The detectable integral moiety may be endogenous to the MSST protein, or may be introduced using recombinant DNA techniques.

The detectable integral moiety becomes more or less accessible to a recognition partner in the presence of ligand compared to the absence of ligand. A “recognition partner” is a molecule, usually a protein, that specifically binds to the detectable integral moiety when it is in the accessible conformation. The recognition partner will vary according to the detectable integral moiety used. For example, where the detectable integral moiety is a protease cleavage site, the recognition partner is a protease that specifically cleaves the protease cleavage site. Where the detectable integral moiety is an antigenic epitope, the recognition partner is an antibody or antibody fragment that specifically finds the antigenic epitope.

Examples of detectable integral moieties will now be described in further detail.

Protease Cleavage Sites as Detectable Integral Moieties

In this embodiment, the conformationally sensitive detectable probe is a protease cleavage site that is introduced into a conformationally sensitive region of an MSST protein. Ligand-induced changes in the conformation of the MSST protein alter its accessibility to a protease specific for the protease cleavage site, and thus its susceptibility to cleavage. For each MSST protein,

In one example, a cleavage site for a highly specific recombinant protease, such as the tobacco etch virus (TEV) protease, is introduced into the third intracellular loop near the cytoplasmic end of TM6 of a GPCR. An alternative site is within the second intracellular loop of a GPCR. Conformational changes induced by ligand binding result in movement of these intracellular loops, thereby altering accessibility of the protease to the cleavage site.

Introduction of Protease Cleavage Sites into a MSST Protein

Protease cleavage sites can be introduced using any suitable conventional methods. In some embodiments it may be desirable to introduce multiple such cleavage sites, e.g., 2 or more, or 3 or more protease cleavage sites.

In general, the MSST protein is modified to have a protease cleavage site introduced at a position so that ligand binding results in an alteration of the accessibility of the cleavage site to protease cleavage, e.g., within a loop that changes in conformation during ligand interaction. FIGS. 20 and 21 provide schematics of the membrane spanning motifs of MSST proteins, and illustrate the extracellular and intracellular regions of such proteins that can be suitable for introduction of a protease cleavage site for use as a conformationally sensitive detectable probe.

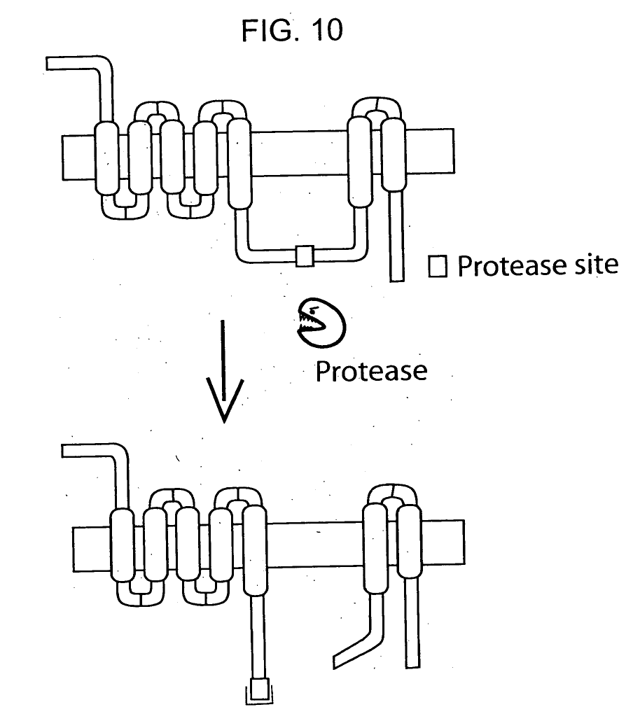

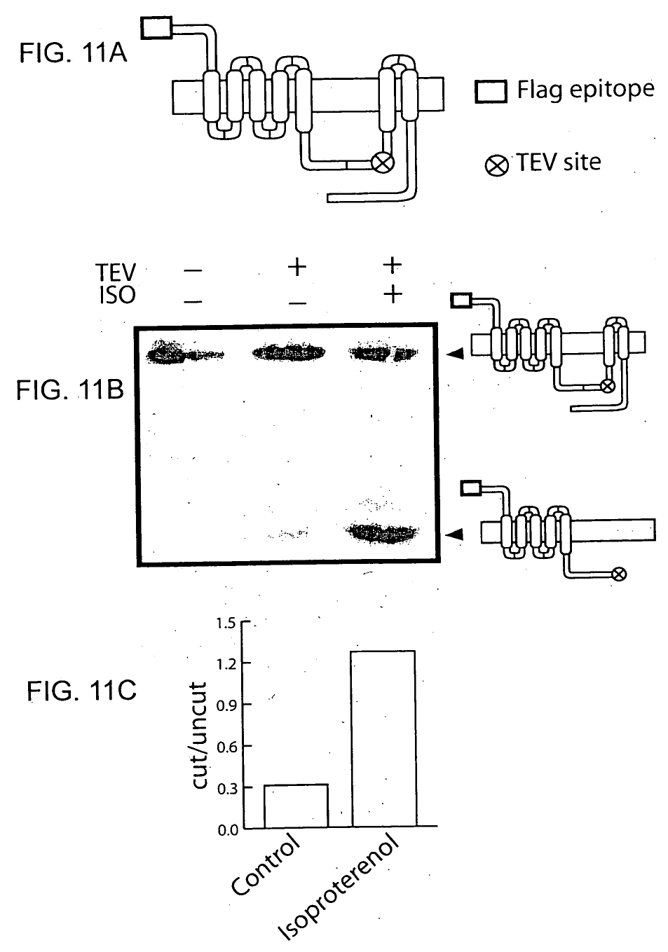

For example, where the MSST protein is a GPCR, the protease cleavage site can be positioned within the third intracellular loop of the GPCR. FIG. 10 provides a schematic of a GPCR having a protease cleavage site within the third intracellular loop and FIGS. 11A-11C show how agonist binding alters protease cleavage.

Protease cleavage site-protease pairs for use in the invention are selected so that cleavage of the modified MSST protein with the protease provides for controlled cleavage of the protein so as to provide for cleavage at a preselected cleavage site(s). In one embodiment, the protease cleavage site-protease pair is selected so that when the MSST protein is in a conformation that provides for accessibility of the cleavage site to protease binding and cleavage, a single cleavage event occurs to generate two cleavage products. In other embodiments, the modified MSST protein contains two protease cleavage site, and may contain three r more cleavage sites. Where the protease cleavage site is introduced into the MSST protein (e.g., the cleavage site is heterologous to the MSST protein), the protease preferentially cleaves at the introduced cleavage site, and cleavage at endogenous sites in the MSST protein are insignificant or undetectable. In some embodiments it may be desirable to modify the MSST protein to remove endogenous sites that act as substrates for a selected protease to enhance specificity and sensitivity of the assay.

Proteolytic cleavage sites are known to those skilled in the art; a wide variety are known and have been described amply in the literature, including, e.g., Handbook of Proteolytic Enzymes (1998) A J Barrett, N D Rawlings, and J F Woessner, eds., Academic Press. Exemplary protease cleavage sites that can be introduced into the modified MSST proteins of the invention include, but are not limited to, tobacco etch virus, furan, and factor Xa proteases.

Further proteolytic cleavage sites include, but are not limited to, an enterokinase cleavage site: (Asp)₄Lys (SEQ ID NO:19); a factor Xa cleavage site: Ile-Glu-Gly-Arg (SEQ ID NO:20); a thrombin cleavage site, e.g., Leu-Val-Pro-Arg-Gly-Ser (SEQ ID NO:21); a renin cleavage site, e.g., His-Pro-Phe-His-Leu-Val-Ile-His (SEQ ID NO:22); (see, e.g., Sommergruber et al. (1994) Virol. 198:741-745).

Detection of Conformational MSST Protein Changes Using Protease as a Conformationally Sensitive Detectable Probe

Detection of protease cleavage products in conformational assays using MSST proteins having a protease cleavage site as a detectable integral moiety can be accomplished in a variety of ways. Exemplary methods for detection of cleavage products include, but are not necessarily limited to: 1) detection of the cleavage product that is produced from the N-terminal portion of the MSST protein; 2) detection of the cleavage product that is produced from the C-terminal portion of the MSST protein; 3) assaying for a new epitope created at an introduced cleavage site following protease action; 4) assaying for the disappearance of an epitope that is present at the cleavage site prior to cleavage; and 5) where the MSST protein is modified to have two protease cleavage sites flanking a detectable polypeptide (e.g., an epitope tag), and detection of the released polypeptide cleavage product. Detection of changes at the protease cleavage site are of particular interest relative to detection of N-terminal or C-terminal cleavage products. Other variations will be readily apparent to the ordinarily skilled artisan.

Epitope Tags

In one embodiment, the MSST protein is modified to include an epitope to facilitate detection (e.g., for detection of a protease cleavage product by detection of an epitope), anchoring of the MSST protein to a substrate (e.g., by binding to an anti-epitope antibody), or both. In general, such modified proteins comprise a heterologous epitope domain. “Heterologous” means that the two elements are derived from two different sources, e.g., the resulting chimeric protein is not found in nature. A variety of epitopes may be used to tag a protein, so long as the epitope (1) is heterologous to the naturally-occurring MSST protein, and (2) the epitope-tagged MSST protein retains at least part and preferably all of the biological activity of the native MSST protein, particularly with respect to the conformational change that occurs upon ligand interaction. Such epitopes may be naturally-occurring amino acid sequences found in nature, artificially constructed sequences, or modified natural sequences.

A variety of artificial epitope sequences are suitable for use as epitope tags in the present invention. In general, any epitope tag useful for tagging and detecting recombinant proteins may be used in the present invention. One such tag, the eight amino acid Flag marker peptide (Asp-Tyr-Lys-Asp-Asp-Asp-Asp-Lys) (SEQ ID NO:1), has a number of features which make it particularly useful for not only detection but also affinity purification of recombinant proteins (Brewer (1991) Bioprocess Technol. 2:239-266; Kunz (1992) J. Biol. Chem. 267:9101-9106). Additional artificial epitope tags include an improved Flag tag having the sequence Asp-Tyr-Lys-Asp-Glu-Asp-Asp-Lys (SEQ ID-NO:2), a nine amino acid peptide sequence Ala-Trp-Arg-His-Pro-Gln-Phe-Gly-Gly (SEQ ID NO:3) referred to as the “Strep tag” (Schmidt (1994) J. Chromatography 676:337-345), poly-histidine sequences, e.g., a poly-His of six residues which is sufficient for binding to IMAC beads, an eleven amino acid sequence from human c-myc recognized by monoclonal antibody 9E10, or an epitope represented by the sequence Tyr-Pro-Tyr-Asp-Val-Pro-Asp-Tyr-Ala-Ile-Glu-Gly-Arg (SEQ ID NO:4) derived from an influenza virus hemagglutinin (HA) subtype, recognized by the monoclonal antibody 12CA5. Also, the Glu-Glu-Phe sequence recognized by the anti-tubulin monoclonal antibody YL½ has been used as an affinity tag for purification of recombinant proteins (Stammers et al. (1991) FEBS Lett. 283:298-302).

Exemplary Assays for Detection of Protease Cleavage Products

As described generally above, detection of conformational changes in MSST proteins by detection of accessibility of a protease cleavage site can be accomplished in a variety of ways. Wherein the MSST protein MSST protein has a single protease cleavage site, the MSST protein is contacted with a candidate agent, and with protease that can cleave the protease cleavage site of the MSST protein. If the candidate agent is, for example, an agonist of the MSST protein, the agent binds to the MSST protein and induces a conformational change that alters the accessibility of the protease cleavage site to cleavage by the protease.

At this point the assay may have up to three different polypeptides present: 1) intact, uncleaved MSST protein (e.g., MSST protein that is not bound by agonist); 2) a protease cleavage product produced from the N-terminal portion of the MSST protein; and 3) a protease cleavage product produced from the C-terminal portion of the MSST protein. In one embodiment, the cleavage products can be detected by western blot analysis (as in FIG. 11B. In another embodiment, the MSST protein is immobilized on a substrate by attachment at the C-terminus (e.g., by binding to an anti-C-terminal MSST protein antibody that is in turn bound to a substrate). Detection of protease cleavage can then be accomplished by detection of a N-terminal MSST protein cleavage product released from the bound MSST protein. Detection of an increased level of N-terminal MSST protein cleavage product in the supernatant relative to a control indicates the candidate agent is a MSST protein ligand that induces a conformational change in the MSST protein. Conversely, candidate agent activity in MSST protein binding can be detected by a decrease in detection of N-terminal MSST protein bound to the substrate.

Alternatively, the MSST protein can be bound to a substrate by the N-terminal end, and a conformational change in the MSST protein due to interaction with the candidate agent can be detected by detection of a released C-terminal MSST protein cleavage product. Conversely, candidate agent activity in MSST protein binding can be detected by a decrease in C-terminal MSST protein bound to the substrate.

In one embodiment, the disappearance of an epitope that is normally present in the MSST protein prior to cleavage can serve as the basis for the assay. For example, the uncleaved MSST protein may have to be modified to have an epitope that can be detected by an antibody, which epitope flanks or encompasses the protease cleavage site. Action of the protease on the cleavage site disrupts the epitope so that it is not detectable in the cleaved MSST protein.

In another embodiment, the action of the protease at the introduced cleavage site is detected by detecting an epitope newly created by the action of the protease. For example, the new epitope can be the newly created C-terminus generated by the protease at the cleavage site.

In another embodiment, the MSST protein is modified to have two protease cleavage sites flanking an epitope tag. Binding of the MSST protein to an agent having, for example, MSST protein agonist activity, causes a conformational change that renders the protease cleavage sites accessible to the protease. Protease cleavage in turn results in liberation of the epitope tag. Detection of the released epitope tag indicates that the MSST protein has undergone a conformational change, and that the candidate agent has activity in binding MSST protein.

All assays can be conducted with an appropriate control, which can be performed in parallel. For example, the level of cleavage product production can be compared to that produced by contacting the MSST protein with a known agonist of the MSST protein.

Immunodetectable Epitopes as Detectable Integral Moieties

In this embodiment, the conformationally sensitive detectable probe is a detectable integral moiety that is an immunodetectable epitope. The epitope, which is present in a conformationally sensitive region of an MSST protein, can be endogenous to the MSST protein, or can be introduced into the protein using recombinant DNA techniques. Ligand-induced changes in the conformation of the MSST protein alter its accessibility of the epitope to binding by a recognition partner, which partner is an antibody or antibody fragment (e.g., Fab).

Suitable immunodetectable epitopes for use in the invention include, but are not necessarily limited to any of the epitope tags described above. Suitable epitope tags are known in the art, and are typically a sequence of between about 6 and about 50 amino acids that comprise an epitope that is recognized by an antibody specific for the epitope. Non-limiting examples of such tags are hemagglutinin (HA; e.g., CYPYDVPDYA (SEQ ID NO; 17)), Flag (e.g., DYKDDDDK (SEQ ID NO:1)), c-myc (e.g., CEQKLISEEDL (SEQ ID NO;18)), and the like.

Suitable recognition partners include antibodies that specifically bind the immunodetectable epitope.

Exemplary Assays for Detection of Detectable Integral Moieties that Comprise Immunodetectable Epitopes

Methods for detecting antibody binding to a substrate are well known in the art. The detection method can involve the use of a detectably labeled antibody (e.g., an antibody or antigen-binding portion of an antibody having a bound detectable chemical label, e.g., a fluorphore). The detectably labeled antibody can bind directly to the immunodetectable epitope (referred to herein as a “primary” antibody), or can bind to an antibody that specifically binds the immunodetectable epitope (e.g., as in a sandwich assay). Antibodies that are specific for anti-immunodetectable epitopes are referred to as “secondary antibodies”. The primary or secondary antibody can be bound to a solid support, or can a solution-based assay. Variations on the configuration of such antibody-based assays are well known in the art.

In one embodiment, FRET between an antibody bound to a non-conformationally sensitive epitope, such as may be on a carboxyl terminus, and an antibody bound to the conformationally sensitive probe is used to detect changes in the conformation of the MSST protein that result in a conformational change at the immunodetectable epitope.

Identification and Design of Therapeutic Compounds

A major asset of the invention is its ability to vastly increase, over current methods, the rate at which compounds can be evaluated for their ability to act as agonists, antagonists, and/or inverse agonists for MSST proteins. As additional MSST protein-encoding genes are identified and characterized, the activity of these proteins in response to various compounds, as well as to methods such as site directed mutagenesis, can be used to gain detailed knowledge about the basic mechanisms at work in these receptors. A fundamental knowledge of the basic mechanisms at work in these receptors will be of great use in understanding how to develop promising new drugs and/or to identify the fundamental mechanisms behind specific signaling pathways.

Identification of Ligands for MSST Proteins such as Orphan GPCRs

An assay system according to the invention can also be used to classify compounds for their effects on a MSST protein for which the endogenous ligand is not known, such as on orphan GPCR receptors, to identify candidate ligands as well as the native ligands for these orphan receptors. Membranes having a modified MSST protein can be exposed to a series of candidate ligands, and the ligands with the ability to induce a conformational change upon the MSST protein identified.

Identification of MSST Proteins Involved in Various Biological Processes

The MSST proteins that are involved in biological response, both normal responses and pathological response (e.g., the biological response to a MSST protein involved in a disease or disorder) can be determined using arrays of the invention. An assay using an array of membranes, each sample of the array having a modified MSST protein, can be exposed to a candidate agent, and any conformational change in the MSST protein(s) detected. This can identify multiple receptors in a high-throughput manner that are involved in the transduction of signals in response to various stimuli. These assays can also be used to determine the specificity of agents by detecting cross-reactivity across different MSST proteins, e.g., different proteins, different protein classes or subclasses, etc.

Automated Screening Methods

The methods of the present invention may be automated to provide convenient, real time, highly parallel, high volume methods of screening compounds for MSST protein ligand activity, or screening for the presence of ligand in a test sample. Automated methods are designed to detect changes in MSST protein activity over time (i.e., comparing the same apparatus before and after exposure to a test sample), or by comparison to a control apparatus that is not exposed to the test sample, or by comparison to pre-established indicia. Both qualitative assessments (positive/negative) and quantitative assessments (comparative degree of translocation) may be provided by the present automated methods.

An embodiment of the present invention includes an apparatus for determining MSST protein response to a test sample. This apparatus comprises means, such as a fluorescence measurement tool, for measuring change in activity of a MSST protein in response to a particular ligand. Measurement points may be over time, or among test and control MSST proteins. A computer program product controls operation of the measuring means and performs numerical operations relating to the above-described steps. The preferred computer program product comprises a computer readable storage medium having computer-readable program code means embodied in the medium. Hardware suitable for use in such automated apparatus will be apparent to those of skill in the art, and may include computer controllers, automated sample handlers, fluorescence measurement tools, printers and optical displays. The measurement tool may contain one or more photodetectors for measuring the fluorescence signals from samples where fluorescently detectable molecules are utilized. Where the conformationally sensitive, detectable probe is a cleavage site, the measurement tool may contain one or more detection reagents for detection of a MSST cleavage product. The measurement tool may also contain a computer-controlled stepper motor so that each control and/or test sample can be arranged as an array of samples and automatically and repeatedly positioned opposite a photodetector during the step of measuring fluorescence intensity.

The measurement tool is preferably operatively coupled to a general purpose or application specific computer controller. The controller preferably comprises a computer program produce for controlling operation of the measurement tool and performing numerical operations relating to the above-described steps. The controller may accept set-up and other related data via a file, disk input or data bus. A display and printer may also be provided to visually display the operations performed by the controller. It will be understood by those having skill in the art that the functions performed by the controller may be realized in whole or in part as software modules running on a general purpose computer system. Alternatively, a dedicated stand-alone system with application specific integrated circuits for performing the above described functions and operations may be provided.

Kits

Also provided by the subject invention are kits for practicing the subject methods, as described above. The kits of the invention at least include one or more of, usually all of: an MSST protein having or modified to contain a conformationally sensitive, detectable probe; and a container (e.g., vial or well) containing the MSST protein or an immobilization phase is to which the MSST protein is attached. The MSST protein can be provided in any suitable form, e.g., in a membrane, e.g., natural, artificial, or surrogate membrane. In one embodiment, the kits of the invention includes at least one candidate agent screening apparatus, where the apparatus comprises an MSST protein and a container as described above. In certain embodiments, the kits further include a positive or negative control, e.g., a positive control, such as a known agonist or antagonist of the MSST protein. Other optional components of the kit include: reagents for detection of the detectable signal of the conformationally sensitive detectable probe (e.g., chemical reagents to facilitate detection of a signal of a detectable chemical label, a protease specific of cleavage of a protease cleavage site, a detectably labeled primary antibody that specifically binds an immunodetectable eptiope, a detectably labeled secondary antibody that specifically binds an antibody specific for an-immunodetectable epitope, and the like), buffers; etc. The various components of the kit may be present in separate containers or certain compatible components may be precombined into a single container, as desired.

Kits of the invention can comprise an apparatus having multiple different MSST proteins for use in screening a candidate agent, which multiple different MSST proteins may be isolated one from another so as to provide separately detectable signals from the conformationally sensitive probes of each MSST protein. Alternatively, the different MSST proteins may be provided in pools. Where a candidate agent modulates activity of a pool of MSST proteins, MSST protein members of such pools can be separately screened using an apparatus where the detectable signals of the MSST proteins can be separately detected.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Methods and Materials

The following methods and materials were used in Examples 1-5 below.

Construction, expression and purification of the β2 adrenergic receptor. Construction, expression and purification of human β2AR were performed as described (Ghanouni, P et al., J Biol Chem 275:3121-3127 (2000)). Mutations Glu224Lys, Cys378Ala, and Cys406Ala (where the first amino acid indicates the native residue, the number indicates the residue position, and the second amino acid represents the amino acid substituted for the native amino acid) were all generated on a background in which all of the lysines in the receptor had been mutated to arginine (Parola, A. L. et al., Anal Biochem 254:88-95(1997)). A sequence coding for the cleavage site for the Tobacco Etch Virus (TEV) protease (Gibco-BRL) was added to the 5′ end of the receptor construct via the linker-adapter method. All mutations were confirmed by restriction enzyme analysis and sequenced. The mutant receptor demonstrated only minor alterations in the general pharmacological properties of the receptor, as assessed by the affinity of the mutant receptor for isoproterenol and alprenolol (K_{I} for ISO=150±40 μM for mutant receptor vs. 210±21 μM for wildtype (Seifert, R., et al., J Biol Chem 273:5109-16(1998)); K_{D} for ALP=4.3±0.6 μM for mutant receptor vs. 1.7±0.9 nM for wildtype (Gether, U. et al., J Biol Chem 270,28268-75 (1995)).

Fluorescent Labeling of Purified β2 Adrenergic Receptor. Purified, detergent soluble receptor was diluted to 1 μM in HS buffer (20 mM Tris, pH 7.5, 500 mM NaCl, 0.1% n-dodecyl maltoside (NDM)) and reacted with 1 μM fluorescein maleimide (FM; Molecular Probes) for 2 h on ice in the dark. The reaction was quenched with the addition of 1 mM cysteine. The receptor was bound to a 250 μl Ni-chelating sepharose column and the column was washed alternately with 250 μl HS buffer and 250 μl NS buffer (20 mM Tris, pH 7.5, 0.1% NDM) for a total of ten cycles to remove free FM. The labeled protein (FM-β2AR) was eluted with HS buffer with 200 mM imidazole, pH 8.0. FM-β2AR was diluted approximately 1:100 in HS buffer for fluorescence measurements. Fluorescence in control samples without receptor was negligible. The labeling procedure resulted in incorporation of 0.6 mol of FM per mol of receptor, based on an extinction coefficient of 83,000 M−1cm−1 for FM and a molecular mass of 50 kDa for the β2AR.

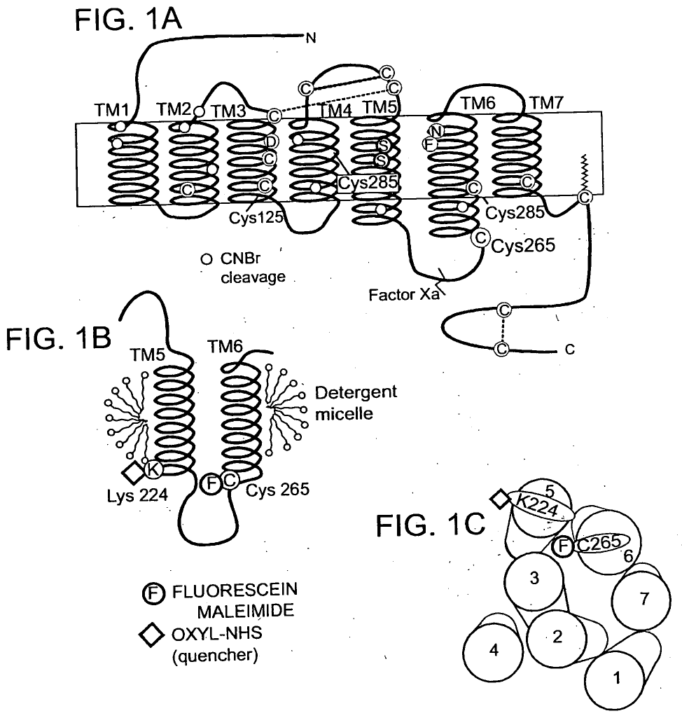

For labeling the Q224K site on the mutant receptor, the sample was split after labeling with FM (1 h) and dialyzed for 1 h at room temperature into a Hepes HS buffer. Half of the sample was treated with 1 mM oxyl-NHS for 1 h on ice. Both the FM alone and the FM+oxyl-NHS samples were then treated with TEV protease (Gibco-BRL) according to the manufacturer's instructions and then washed on a Ni-chelating sepharose column as above. Equivalent amounts of FM− and FM+oxyl-NHS-labeled receptor, as confirmed by protein assay (Bio-Rad DC Kit), were thus prepared for comparison. The TEV protease site at the N-terminus of the receptor allowed us to remove any probe located at the N-terminus after labeling the receptor with an amine-reactive tag. The location of the FM labeling site at Cys265 in both the wildtype and mutant receptors was verified by peptide mapping with protease factor Xa and cyanogen bromide. Cleavage sites are as indicated in FIG. 1.

Fluorescence spectroscopy. Experiments were performed on a SPEX Fluoromax spectrofluorometer with photon counting mode using an excitation and emission bandpass of 4.2 nm. Approximately 25 pmol of FM-labeled O₂ adrenergic receptor were used in 500 μl of HS buffer. Excitation was at 490 nm and emission was measured from 500 to 599 nm with an integration time of 0.3 s/nm for emission scan experiments. For time course experiments, excitation was at 490 nm and emission was monitored at 517 nm. For studies measuring ligand effects, no difference was observed when using polarizers in magic angle conditions. Unless otherwise indicated, all experiments were performed at 25° C. and the sample underwent constant stirring. Fluorescence intensity was corrected for dilution by ligands in all experiments and normalized to the initial value. All of the compounds tested had an absorbance of less than 0.01 at 490 and 517 nm in the concentrations used, excluding any inner filter effect in the fluorescence experiments.

Fluorescence lifetime determination. Fluorescence lifetime measurements of the FM-labeled β2 adrenergic receptor were carried out using a PTI Laserstrobe fluorescence lifetime instrument. Measurements were taken at 25° C., using 490 nm excitation pulses (full width half maximum (FWHM)˜1.4 ns) to excite the samples, and emission was monitored through a combination of three >550 nm long pass filters. Measurements used 225 μl of a 5 μM sample placed in a 4×4 mm cuvette, and represent 3 average shots of 5 shots per point, collected in 150 channels. The fluorescence decays were fit to a single exponential using the commercial PTI program.

Quenching of fluorescence. To quench the fluorescence, FM was diluted to 1 μM in HS buffer. The dye was diluted into 375 μl of a buffer containing 20 mM HEPES, pH 7.5, and 0.1% NDM. Experiments were performed at the indicated concentration of potassium iodide, freshly made in 10 mM Na₂S₂O₃, while the total salt concentration was maintained at 250 mM with potassium chloride in all experiments. Potassium iodide and potassium chloride at concentrations up to 250 mM do not alter the ligand binding properties of the β2AR (Gether et al. (1995) J. Biol. Chem. 270:28268-75). For nitroxide quenching, receptor was diluted into HS buffer. Experiments were performed at the indicated concentration of nitroxide fatty acids (Molecular Probes), while maintaining total fatty acid concentration at 100 μM with stearic acid. After each addition of quencher, samples were thoroughly mixed, incubated for 10 min (KI) or 5 min (nitroxides), and fluorescence was recorded by exciting at 490 nm and performing an emission scan from 500-599 nm.

Data were plotted according to the Stern-Volmer equation, Fo/F=1+Ksv(KI), where Fo/F is the ratio of fluorescence intensity in the absence and presence of KI, and Ksv is the Stern-Volmer quenching constant. The Ksv values thus obtained were then used with the measured fluorescence lifetimes (τₒ) to determine the bimolecular quenching constant, kq (Ksv=kq·τₒ) (Lakowicz, J. R. (1983) Plenum Press, N.Y.). For quenchers, a time scan was initiated after the emission scan and 100 μM (−)-isoproterenol was added after 2 min. At 10 min, 20 μM (−)-alprenolol was added and the extent of reversal determined. The quenchers used did not alter the ability of (−)-isoproterenol or (−)-alprenolol to compete with (3H)DHA.

### Example 1

### Effect of Full and Partial Agonists on Fluorescence of FM-β2AR Correlates with the Biological Properties of the Agonists

The effect of full and partial agonists on the fluorescence of FM-β2AR correlated with the biological properties of the agonists. Only Cys265 was labeled when purified, detergent solubilized β2AR (1 μM) is reacted with fluorescein maleimide at a 1:1 stoichiometry. This polar fluorophore does not label transmembrane cysteines and the two other potentially accessible cysteines in the carboxyl terminus (FIG. 1A) form a disulfide bond during purification; The specificity of labeling was confirmed by peptide mapping studies with factor Xa (which cleaves only in the third intracellular loop) and cyanogen bromide (which cleavage at methionines, shown in FIG. 1A). When FMβ2AR is cleaved with factor Xa fluorescence labeling is only observed on the carboxyl terminal half of the protein. Following cleavage of FMβ2AR with cyanogen bromide labeling is localized to a 7 kDa peptide representing a portion of the third intracellular loop containing Cys 265 (data not shown). Labeling of the β2AR with fluorescein did not alter ligand binding or G protein coupling in a reconstitution assay (data not shown).

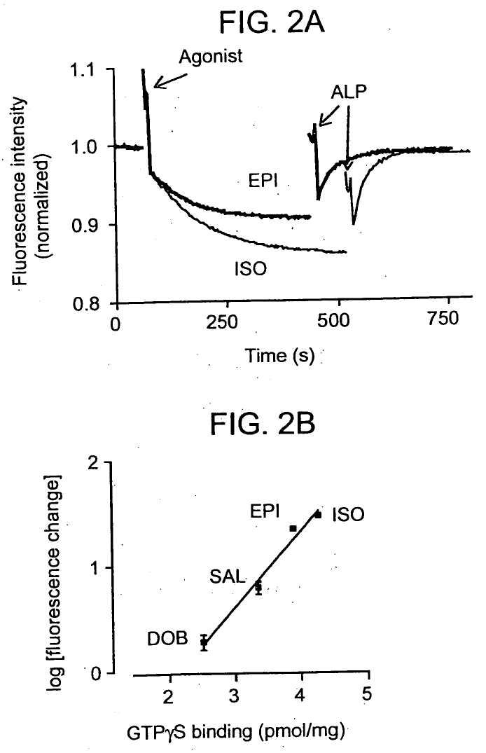

The fluorescence properties of FM-β2AR were examined by monitoring fluorescence as a function of time. As illustrated in FIG. 2A, the change in intensity of FM-β₂AR in response to the addition of the full agonist (−)-isoproterenol (ISO) and the strong partial agonist epinephrine (EPI) was reversed by the neutral antagonist (−)-alprenolol (ALP). All data represent experiments performed in triplicate. In most experiments, the ALP reversal was used to quantitate the magnitude of the agonist-induced change. The ALP reversal was found to be the most consistent measure for comparison of agonist-induced conformational changes because ALP reversal occurs over a shorter period of time relative to agonist responses and therefore is less subject to non-specific effects on fluorescence intensity (e.g., photobleaching, receptor denaturation) that affect the baseline. ALP alone did not induce any changes in fluorescence and treatment with ligands did not cause a change in the wavelength of maximum emission (data not shown). The partial agonists epinephrine (EPI), salbutamol (SAL) and dobutamine (DOB) produce progressively smaller changes in receptor fluorescence.

The agonist and partial agonist effects on the intensity of FM-β₂AR were compared with an assay of biological efficacy (GTPγS binding). FM-β₂AR was treated with different agonists and the change in fluorescence was measured at a time equal to 5 times the calculated t½ for each drug. All agonists were used at 100 mM in order to ensure saturation of the receptors and eliminate the effect of variations in agonist affinities. The ability of these ligands to stimulate GTPγS binding in a γ₂AR-Gαs fusion protein was determined as previously described (Lee et al. (1999) Biochemistry 38:13801-9). All data represent experiments performed in triplicate. The magnitude of the effect of agonists on the fluorescence intensity of FM-β2AR correlates with the biological efficacy of these drugs in β2AR-mediated activation of Gs in membranes (FIG. 2B).

These experiments verify that fluorescence intensity changes in FM-β2AR reflect biologically relevant, ligand-induced conformational changes.

### Example 2

### Kinetics of Agonist-Induced Conformational Change

Rhodopsin has long been used as a model system for direct biophysical analyses of GPCR activation because of its natural abundance, inherent stability, and spectroscopically defined activation scheme (Sakmar, T. P., Prog Nucleic Acid Res Mol Biol 59:1-34 (1998)). The recent crystal structure of bovine rhodopsin (Palczewski, K. et al., Science 289, 739-45 (2000)) provides the first high-resolution picture of the inactive state of this highly specialized GPCR. While the general features of this structure presumably apply across the broad family of GPCRs, the mechanism of rhodopsin activation is unique among GPCRs because of the presence of a covalent linkage between the receptor and its ligand, retinal. Thus, the dynamic processes of agonist association and dissociation common to the GPCRs for hormones, neurotransmitters, and other sensory stimuli are not part of the activation mechanism of rhodopsin. In contrast to rhodopsin, the β2 adrenergic receptor is activated by a functionally broad spectrum of diffusible ligands.

This difference between rhodopsin and the β2AR is reflected in the rate of agonist-induced structural changes. Conformational changes induced in detergent-solubilized preparations of rhodopsin by light activation were very rapid, occurring with a t½ of milliseconds (Arnis et al., J Biol Chem 269, 23879-81 (1994); Farahbakhsh, et al., Science 262, 1416-9 (1993)). In contrast, as shown in FIGS. 2A-2B, agonist activation of the β₂AR was slow, despite the rapid on-rate of agonist binding (t½˜20 sec) as calculated from the agonist affinity, the off-rate estimated from the alprenolol (ALP) reversal of the agonist effect (FIG. 2A) and the concentration of agonists used in these experiments (100 μM)). Under these conditions, the on-rate of agonist was comparable to the more rapid rate of reversal of the agonist effect by the antagonist alprenolol (t½ at 25° C.=22.8±3.6 s, Mean±S.E.M., n=3).

The same slow rate of agonist-induced conformational change was also observed with a different fluorescent reporter on Cys125 in TM3 and on Cys285 in TM6 of the β2AR (FIG. 1A) (Gether, U., Lin, S., Ghanouni, P., Ballesteros, J. A., Weinstein, H. & Kobilka, B. K. (1997) Embo J 16, 6737-47), and Salamon and colleagues observed a similar rate of agonist induced conformational changes in the α-opioid receptor analyzed by surface plasmon resonance spectroscopy (Salamon, Z. et al., Biophys J 79:2463-74 (2000)). Thus, agonist binding precedes the conformational change. The rate of conformational change is temperature dependent, with the rate at 37° C. approximately 3 times that at 25° C. (data not shown). The slow, temperature dependent rate of conformation change and the rapid reversal suggests that the active state is a relatively high energy state which may be reached through one or more intermediate states, as illustrated in Equation 1:

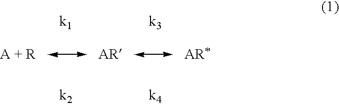

where R is the inactive receptor, R′ is the agonist bound, inactive receptor and R* is the active receptor, k3 is predicted to be slow relative to k1, k2 and k4. Moreover the agonist binding site in R′ may not be identical to the binding site in R*. The ligand binding site for the β2AR has been well characterized by mutagenesis studies and lies relatively deep in the transmembrane domains (FIG. 1A). Without being held to theory, the difference in the rate of conformation change between rhodopsin and the β2AR can be attributed to the need for the ligand to diffuse into the binding pocket and the smaller energy associated with agonist binding.

### Example 3

### Agonist-Induced Movement of FM Bound to Cys265 Relative to Molecular Landmarks

To characterize the agonist-induced structural changes in the G protein coupling domain containing Cys265, agonist-induced changes in the interaction of FM-β2AR with a variety of fluorescence quenchers was examined.

The results of these experiments were interpreted in the context of a three dimensional model of the β2AR based on the recent crystal structure of rhodopsin in the inactive state. Based on a simplified model viewed from the cytoplasmic surface of the receptor, we would predict that in the absence of agonist, fluorescein bound to Cys265 would be facing the interior of a bundle of helices formed by the cytoplasmic extensions of TM3, TM5 and TM6 (FIG. 1C).

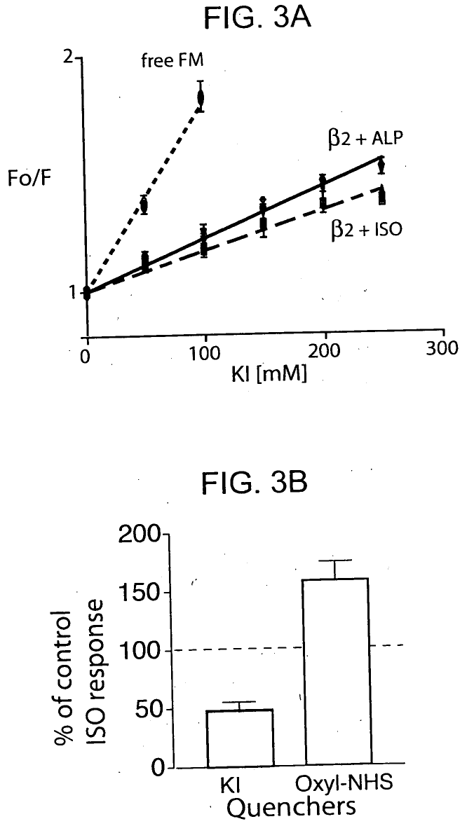

The accessibility of the water-soluble quencher potassium iodide to the fluorescein bound to Cys265 was then determined (FIG. 3A). Potassium iodide (KI) was added to fluorescein maleimide reacted with cysteine, to labeled receptor incubated with 20 mM (−)-alprenolol, and to labeled receptor incubated with 100 mM (−)-isoproterenol. Fluorescence was measured and plotted as described in Methods. The quenching constant Kₛᵥ was 7.9±0.4 M⁻¹ for fluorescein alone, 2.19±0.06 M⁻¹ for labeled receptor incubated with (−)-alprenolol, and 1.66±0.06 M⁻¹ for labeled receptor incubated with (−)-isoproterenol. The difference between isoproterenol and alprenolol was significant (p<0.05, unpaired t test). There was no difference in Kₛᵥ between buffer alone and alprenolol treatments. All values are Mean±S.E.M., n=3. The results are shown in FIG. 3A.

The effect of quenchers KI and Oxyl-NHS on the magnitude of the ISO-induced decrease in fluorescence was also determined (FIG. 3B). “% of control ISO response” was calculated using the formula [100(ISO induced change in fluorescence in the presence of quencher)/(ISO induced change in fluorescence in the absence of quencher)]. For the aqueous quencher KI, the ISO-induced change in fluorescence in the presence of 250 mM KI was less than that in the presence of 250 mM KCl (55.4±8.3% of control ISO response). (In contrast to the aqueous quencher KI, covalent binding of the spin-labeled quencher Oxyl-NHS to K224 in TM5 increased the magnitude of the ISO response relative to the control (158±8% of control ISO response), see below). In these experiments, the magnitude of the ALP reversal of the ISO-induced change in fluorescence was used as a measure of the magnitude of the ISO response. The results are shown in FIG. 3B. All values are Mean±S.E.M., n=3.

As represented in the Stern-Volmer plot (FIG. 3A), steady-state fluorescence quenching by KI is much lower for fluorescein bound to the receptor when compared to fluorescein maleimide bound to free cysteine in solution. This indicates that the fluorescein site on the receptor is relatively inaccessible to the water soluble quencher KI, as expected based on the predicted position of the fluorescein bound to Cys265 (FIG. 1C).

To determine the effect of agonist on KI quenching, we measured the fluorescence lifetimes of FM-β2AR in the presence ISO and ALP, which permitted us to calculate the bimolecular quenching constant (kq Ksv/τₒ) using the average value of the lifetime of FM-β2AR in the presence of either ISO (kq=0.45±0.01×10−9 M−1s−1) or ALP (kq=0.51±10.01×10−9 M−1s−1). There was no difference between the extent of KI quenching in the ligand-free or ALP-bound receptor. However, the lower kq in the ISO bound state clearly shows that the fluorescein label on the β2AR was less accessible to the water-soluble quenching reagent KI in the presence of the agonist ISO (Dunham and Farrens J Biol Chem 274:1683-90 (1999)). As a result, the magnitude of the ISO-induced change in fluorescence in the presence of 250 mM KI was smaller than in the presence of 250 mM KCl (FIG. 3B). Thus, ISO induces a conformational change that enhances the intra-receptor quenching of FM bound to Cys265, but reduces access of Cys265 to exogenous, aqueous quencher KI. The burial of Cys265 away from the aqueous milieu could be accomplished by a movement of TM6 toward the membrane (FIG. 1B) and/or by a movement of TM6 that would bring Cys265 closer to either TM3 or TM5 (FIG. 1C).

### Example 4

### Agonist-Induced Movement of Cys265 Relative to Lys224

To distinguish between the movement of Cys265 toward either TM3 or TM5, a modified β2AR that permits site-specific attachment of an amine-reactive, spin-labeled quencher at the cytoplasmic border of TM5 was generated (FIG. 1C). In order to position the quencher at the base of TM5, the template β2AR was used in which all of the lysines have been replaced by arginine (Parola et al., Anal Biochem 254, 88-95 (1997)) and changed Glu224 to lysine. This mutant was purified and studied the interaction between FM at Cys265 and oxyl-NHS at Lys224.

While the baseline quenching of FM on Cys265 with oxyl-NHS bound to Lys224 was less that 10%, the effect of ISO on decreasing of FM fluorescence intensity (as reflected in the magnitude of the ALP reversal) was enhanced by more than 50% with the quencher bound to Lys224 (FIG. 3B). Since the effect of this quencher was distance dependent, the increase in the extent of quenching reflects an agonist-induced conformational change that brings these regions of TM6 and TM5 closer together.

### Example 5

### Agonist Induces Movement of FM Bound to Cys265 Relative to a Lipophilic Quencher in the Detergent Micelle

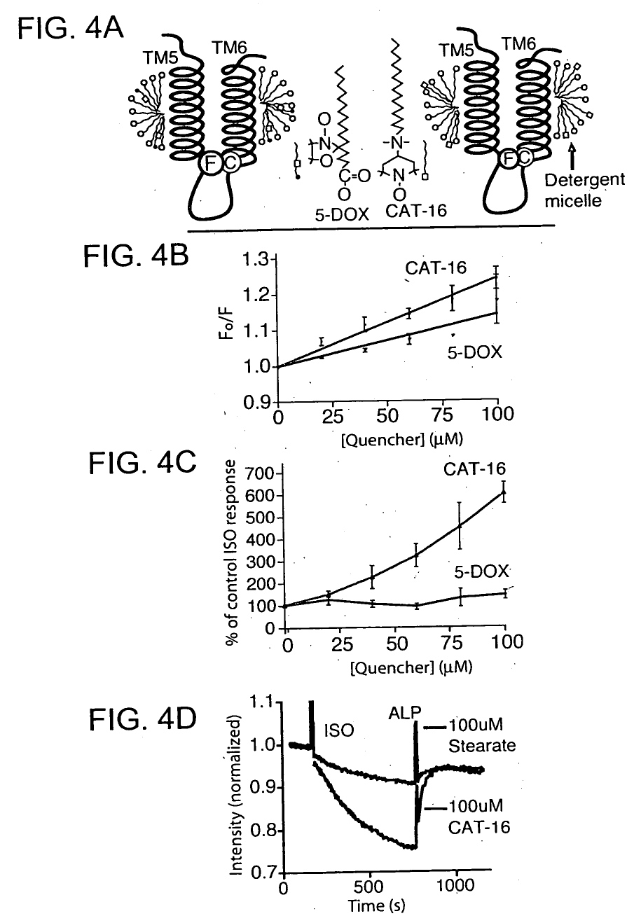

Due to the location of the fluorophore close to the predicted protein-lipid interface (FIG. 1B) of TM6, the interaction between the fluorophore and nitroxide spin-labeled-fatty acids which partition into the detergent micelle was used to observe relative motion between the Cys265 and the micelle (FIG. 4A). FIG. 4A is a schematic depicting the structure of CAT-16 and 5-doxyl stearate (5-DOX), as well as the putative location of these quenching groups in the micelle. The quenching group on CAT-16 is localized on the polar surface of the micelle. The quenching group on 5-DOX is located within the hydrophobic core of the micelle.

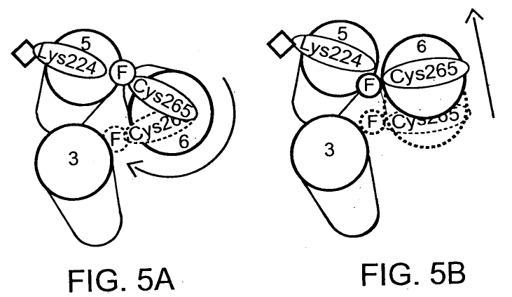

FIG. 4B provides a Stern-Volmer plot depicting the extent of quenching of FM-b2 AR by increasing concentrations of CAT-16 or 5-DOX. Quenchers were added to labeled receptor and fluorescence was measured and plotted as in FIG. 3 and Methods. The total lipid concentration was kept constant at 100 mM with stearic acid. The quenching constant Ksv was 2.4±0.1 mM⁻¹ in the presence of CAT-16 and 1.4±0.2 mM⁻¹ in the presence of 5-DOX. FIG. 5C shows the differing effects of CAT-16 and 5-DOX on agonist-induced fluorescence change of FM-b2 AR. The extent of response to (−)-isoproterenol is presented as a % control ISO response, calculated as in FIG. 3. FIG. 5D is an example of the experiments used to generate the ratios in FIG. 4c. In this example, FM-β2 AR was incubated with either 100 mM CAT-16 or with 100 mM stearic acid. The response to agonist was monitored as described for the experiment depicted in FIG. 2. In the presence of the quencher CAT-16, (−)-isoproterenol induced a 24.2±0.3% decrease in fluorescence versus 4.1±0.6% in the presence of the stearic acid. All values are Mean±S.E.M., n=3.

Because of their ability to quench the excited state of a variety of fluorophores in a distance-dependent manner, these spin-labeled fatty acid derivatives have been used extensively to study the distribution, location and dynamics of fluorescently tagged proteins and lipids (Matko J. et al, Biochemistry 31, 703-11 (1992)). Fatty acid derivatives with spin labels at two different locations along the carbon chain were examined (FIG. 4A) and observed the best quenching of fluorescein by CAT-16, which has a charged spin label on the head group of the fatty acid (FIG. 4B). The magnitude of the change in fluorescence intensity of FM-β2AR in response to the agonist ISO is dramatically increased in the presence of CAT-16 compared to the control fatty acid stearate (FIG. 4c). This effect was not observed with 5-DOX (FIG. 4C). For example, 100 μM 5-DOX quenched baseline fluorescence by 12% (FIG. 4B), but had no significant effect on the magnitude of the agonist-induced change in fluorescence (FIG. 4C). In contrast, 50 μM CAT-16 produced a similar (˜12%) quenching in baseline fluorescence (FIG. 4b), but increased the magnitude of the agonist-induced fluorescence change by more than two fold (FIG. 4c). This indicates that ISO induces a conformational change at Cys265 which brings the fluorophore closer to the nitroxide spin label of CAT-16 in the detergent micelle border, but not significantly closer to nitroxide spin label in 5-DOX, which would be buried within the hydrophobic core of the micelle. According to the models shown in FIG. 4a and FIG. 5, a piston-like movement of TM6 into the detergent micelle would bring fluorescein closer to the quenchers on both 5-DOX and CAT-16, but a clockwise rotation of TM6 and/or a tilting of TM6 would bring fluorescein closer to CAT-16 without significantly changing its position relative to 5-DOX.

### Examples 6-9

### Functionally Different Agonists Induce Distinct Conformations in the G Protein Coupling Domain of β2AR

Methods and Materials

The following methods and materials were used in Examples 6-9.

Fluorescence spectroscopic studies of the β₂AR. Construction, expression and purification of human β₂AR were performed as described (Gether, et al. (1995) J Biol Chem 270(47), 28268-75). For labeling, purified, detergent-solublized wild-type receptor was diluted to 1 μM in HS buffer (20 mM Tris, pH 7.5, 500 mM NaCl, 0.1% n-dodecyl maltoside (NDM)) and reacted with 1 μM fluorescein maleimide (FM; Molecular Probes) for 2 h on ice in the dark. The reaction was quenched with the addition of 1 mM cysteine. The receptor was bound to a 250 μl Ni-chelating sepharose column and the column was washed alternately with 250 μl HS buffer and 250 μl NS buffer (20 mM Tris, pH 7.5, 0.1% NDM) for a total often cycles to remove free FM. The labeled protein (FM-β₂AR) was eluted with HS buffer with 200 mM imidazole, pH 8.0. FM-β₂AR was diluted approximately 1:100 in HS buffer for fluorescence measurements. Fluorescence in control samples without receptor was negligible.

The stoichiometry of labeling was determined by measuring absorption at 490 nm and using an extinction coefficient of 83,000 M⁻¹ cm⁻¹ for FM and a molecular mass of 50 kDa for the β₂AR. The labeling procedure resulted in incorporation of 0.6 mol of FM per mol of receptor. Fluorescence spectroscopy experiments were performed on a SPEX Fluoromax spectrofluorometer with photon counting mode using an excitation and emission bandpass of 4.2 nm. Approximately 25 pmol of FM-labeled P2 adrenergic receptor was diluted into 500 μl of 200 mM Tris, pH 7.5, 500 mM NaCl, 0.1% NDM, 100 mM mercaptoethanolamine (MEA). Excitation was at 490 nm and emission was measured from 500 to 599 nm with an integration time of 0.3 s/nm for emission scan experiments.

For time course experiments, excitation was at 490 nm and emission was monitored at 517 nm. For anisotropy studies, fluorescence intensities were measured with excitation and emission polarizers in horizontal (H) and vertical (V) combinations. The G factor was calculated from the ratio of the intensities (I) of I_{HV}/I_{HH} and the anisotropy (r) was calculated from

$$r = ( \frac{{I}_{VV} - {GI}_{VH}}{{I}_{VV} + 2 {GI}_{VH}} ) .$$

For studies measuring ligand effects, no difference was observed when using polarizers in magic angle conditions. Unless otherwise indicated, all experiments were performed at 25° C. and the sample always underwent constant stirring. The volume of the added ligands was ≦1% of total volume, and fluorescence intensity was corrected for this dilution in all experiments shown. All of the compounds tested had an absorbance of less than 0.01 at 490 and 517 nm in the concentrations used, excluding any inner filter effect in the fluorescence experiments.

Fluorescence lifetime analysis of fluorescein labeled β₂AR. To determine fluorescence lifetimes, approximately 250 pmol FM-β₂AR was diluted in 1.5 ml of 200 mM Tris, pH 7.5, 500 mM NaCl, 0.1% NDM, 100 mM MEA and incubated for 10 min at 25° C. with or without ligand. Fluorescence lifetimes were measured using a frequency-domain 10 GHz fluorometer equipped with Hamamatsu 6-μm microchannel plate detector (MCP-PMT) as previously described (Laczko, et al. (1990) Rev. Sci. Instrum. 61, 2331-2337). The instrument covered a wide frequency range (4-5000 MHz), which allowed detection of lifetimes ranging from several nanoseconds to a few picoseconds. Samples were placed in a 10-mm path-length cuvette. The excitation was provided by the frequency-doubled output of a cavity-dumped pyridine-2 dye laser tuned at 370 nm synchronously pumped by a mode-locked argon ion laser. Sample emission was filtered through Corning 3-72 and 4-96 filters. For the reference signal, DCS in methanol (463 ps fluorescence lifetime) was observed through the same filter combination.

The governing equations for the time-resolved intensity decay data were assumed to be a sum of discrete exponentials as in I(t)=IₒΣᵢαᵢe^{t/τ}^{ᵢ}, where I(t) is the intensity decay, αᵢ is the amplitude (pre-exponential factor) and τᵢ is the fluorescence lifetime of the i-th discrete component; or a sum of Gaussian distribution functions as in the equation I(t)=IₒΣᵢαᵢτe^{t/τ} and

$${α}_{i} ( τ ) = \frac{1}{σ \sqrt{2} π} {ⅇ}^{- \frac{1}{2} {( \frac{t - τ}{σ} )}^{2}}$$

where τ is the center value of the lifetime distribution and σ is the standard deviation of the Gaussian, which is related to the full width at half-maximum by 2.354 σ. In the frequency domain, the measured quantities at each frequency ω, are the phase shift (Øω) and demodulation factor (m_{ω}) of the emitted light versus the reference light.

Fractional intensity, amplitude, and lifetime parameters were recovered by a non-linear least squares procedure using the software developed at the Center for Fluorescence Spectroscopy. The measured data were compared with calculated values (Ø_{cω},m_{cω}) and the goodness of fit was characterized by

$${Χ}_{R}^{2} = \frac{1}{υ} {\sum }_{ω} {( \frac{{φ}_{ω} - {φ}_{c ω}}{δφ} )}^{2} + \frac{1}{υ} {\sum }_{ω} {( \frac{{m}_{ω} - {m}_{c ω}}{δ m} )}^{2} ,$$

where ν is the number of degrees of freedom and δØ and δm are the uncertainties in the measured phase and modulation values, respectively. The sum extends over all frequencies (ω).

### Example 6

### Using Fluorescence Lifetime Spectroscopy to Study Ligand-Induced Conformational Changes in the β₂AR

The β2AR was purified and labeled at Cys265 with fluorescein maleimide to generate FM-β₂AR as previously described. Ligand-dependent changes in fluorescence lifetime of FM-β₂AR were examined in an effort to identify the existence of agonist-specific conformational states. Fluorescence lifetime analysis can detect discrete conformational states in a population of molecules, while fluorescence intensity measurements reflect the weighted average of one or more discrete states.

Based on the observed changes in steady-state fluorescence intensity, it was predicted that ligand-induced conformational changes in the receptor would alter the fluorescence lifetime of the fluorophore. Fluorescence lifetime, τ, refers to the average time that a fluorophore which has absorbed a photon remains in the excited state before returning to the ground state. The lifetime of fluorescein (nanoseconds) is much faster than the predicted off-rate of the agonists we examined (τs-ms), and much shorter than the half-life of conformational states of bacteriorhodopsin (μs) (Subramaniam, et al. (2000) Nature 406(6796), 653-7), rhodopsin (ms) (Farahbakhsh, et al. (1993) Science 262(5138), 1416-9; Arnis, et al. (1994) J Biol Chem 269(39), 23879-81) or of ion channels (μs-ms) (Hoshi, et al. (1994) J Gen Physiol 103(2), 249-78). Therefore, lifetime analysis of fluorescein bound to Cys265 is well-suited to capture even short-lived, agonist-induced conformational states.

### Example 7

### Antagonist Binding Narrows the Distribution of Fluorescence Lifetimes

Data from fluorescence lifetime experiments on FM-β₂AR bound to different drugs at equilibrium were analyzed in two ways. Traditionally, fluorescence decays are fit to single and multiple discrete exponential functions and the best fit determined by χ² analysis. In this analysis, the observed fluorescence decay was resolved into one or more exponential components, with each component, i, being described by τᵢ and τᵢ, where τᵢ represents the fractional contribution of τᵢ to the overall decay. The best fit to single or multiple components was determined by χ² analysis. If different agonists induce a single active state, then the fluorescence lifetime associated with that state (τ_{R*}) should be the same for different drugs and only the fractional contributions (τ_{DRUG}) should differ. However, if there are agonist-specific conformational states we should observe unique, agonist-specific lifetimes (e.g. τ_{ISO}, τ_{SAL}, and τ_{DOB}).

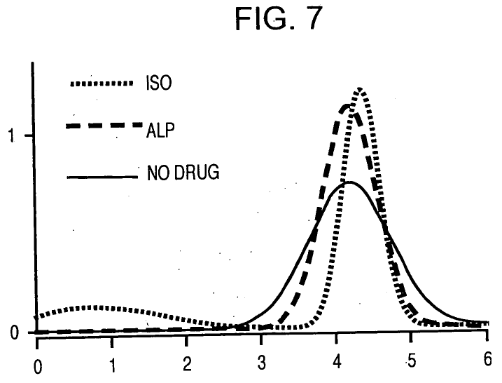

This discrete component analysis assumes that the receptor exists in one or a few rigid protein conformations and does not accurately reflect the dynamic nature of proteins. Proteins that are functionally in a single conformational state actually undergo small conformational fluctuations around a minimum energy state (Frauenfelder, et al. (1991) Science 254(5038), 1598-603) and these small structural perturbations can lead to small changes in the environment around an attached fluorophore. These perturbations are thought to reflect local unfolding reactions within the three dimensional structure of proteins (Freire, E. (2000) Proc Natl Acad Sci USA 97(22), 11680-2). Such flexibility in protein structure can be modeled using fluorescence lifetime distributions (Gratton, et al. (1989) in Fluorescent Biomolecules: Methodologies and Applications (Jameson, D. M., ed), pp. 17-32, Plenum Press, New York), wherein the width of the distributions reflects the conformational flexibility of the protein (FIG. 7). The mobility of fluorescein relative to the receptor is minimal, as determined by its high measured anisotropy (r=0.30±0.02, n=3), and therefore would be expected to contribute little to the width of the lifetime distribution. Thus, the width of the distribution can be attributed to conformational flexibility in the receptor itself.

Lifetime analysis of unliganded FM-β₂AR reveals a single, flexible state. This is indicated by both the single, broad Gaussian distribution of lifetimes centered around 4.2 ns (FIG. 7, “NO DRUG” trace), and the discrete component analysis, where the fluorescence decay rate of FM-β₂AR in the absence of any drug is best fit by a single exponential function (Table 1). Binding of the neutral antagonist ALP to FM-β₂AR does not significantly change the fluorescent lifetime (Table 1), but does narrow the distribution of lifetimes (FIG. 7, “ALP” trace), suggesting that ALP stabilizes the receptor and reduces conformational fluctuations. This interpretation is consistent with the results of experiments demonstrating that the β₂AR is more resistant to protease digestion when bound to ALP (Kobilka, B. K. (1990) J Biol Chem 265(13), 7610-8).

TABLE 1
Fluorescent lifetime data for FM-β₂AR
in the presence and absence of
drugs fit to discrete exponential functions.

| | τ₁ (nsec) | τ₂ (nsec) | α₂ | χ² |
|---|---|---|---|---|
| NO DRUG | 4.22 ± 0.02 | — | — | 2.9 ± 0.4 |
| ALP | 4.21 ± 0.01 | — | — | 3.1 ± 0.8 |
| ISO | 4.30 ± 0.01 | 0.77 ± 0.05 | 0.19 ± 0.03 | 3.3 ± 1.0 |
| SAL | 4.35 ± 0.02 | 1.45 ± 0.16 | 0.08 ± 0.01 | 2.0 ± 0.2 |
| DOB | 4.36 ± 0.01 | 1.68 ± 0.3 | 0.07 ± 0.01 | 1.8 ± 0.4 |

### Example 8

### Agonists and Partial Agonists Induce Distinct Conformations

Unexpectedly, binding of the full agonist ISO promotes conformational heterogeneity. In the presence of saturating concentrations of ISO, FM-β₂AR has two distinguishable fluorescence lifetimes (FIG. 7 and Table 1) representing at least two distinct conformational states. The long lifetime component is only slightly longer than the lifetime observed in the absence of drugs; however, the distribution is narrower than that observed in the presence of the antagonist ALP (FIG. 7, compare “ISO” and “ALP” traces). In contrast, the distribution of the short lifetime component observed in the presence of ISO is relatively broad, suggesting that there is considerable flexibility around Cys265 in this agonist-induced conformation.

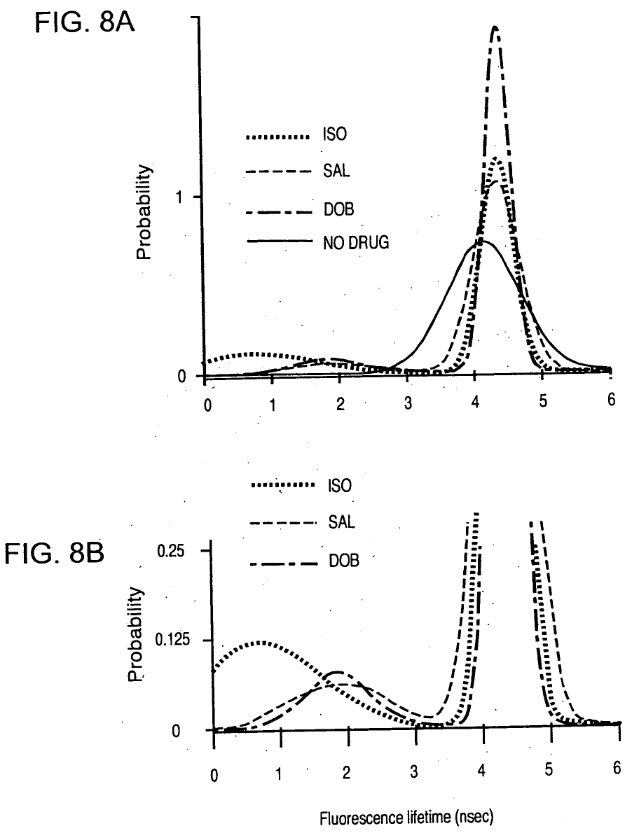

The effect of the partial agonists salbutamol (SAL) and dobutamine (DOB) on the fluorescence lifetime of FM-β₂AR was next examined. Similar to ISO, we observed two lifetimes when the receptor was bound to saturating concentrations of SAL and DOB (Table 1 and FIGS. 8A-8B). The long lifetime component found in the presence of these two partial agonists is indistinguishable from that observed in the ISO-bound receptor; however, the short lifetime component found in both the SAL- and DOB-bound receptor is statistically different from that for the ISO-bound receptor. A strong correlation was observed between a reduction in fluorescence intensity of FM bound to Cys265 and drug efficacy, and shortening of the average fluorescence lifetime is associated with a reduction in fluorescence intensity. Therefore, the short lifetime, found only in the presence of agonists, likely represents the G protein activating conformation of FM-β₂AR.

The different short lifetimes for the full agonist (ISO) and the partial agonists (SAL and DOB) indicate different molecular environments around the fluorophore and therefore represent different, agonist-specific active states. The narrowing and rightward shift of the long lifetime component following binding of both agonists and partial agonists indicate that this lifetime also reflects an agonist-bound state, but most likely represents a more abundant intermediate state that would not be expected to alter greatly the intensity of FM bound to Cys265. It is possible that the number of conformations that we observe in these experiments represent only a few of the possible conformations that can be stabilized by drugs. Moreover, while the overlapping short lifetime distributions of SAL and DOB (FIG. 8B and Table 1) suggest that they induce similar conformations, it is possible that a conformationally sensitive probe positioned elsewhere on the receptor could distinguish between DOB- and SAL-bound receptors states.

### Example 9

### Models of GPCR Activation

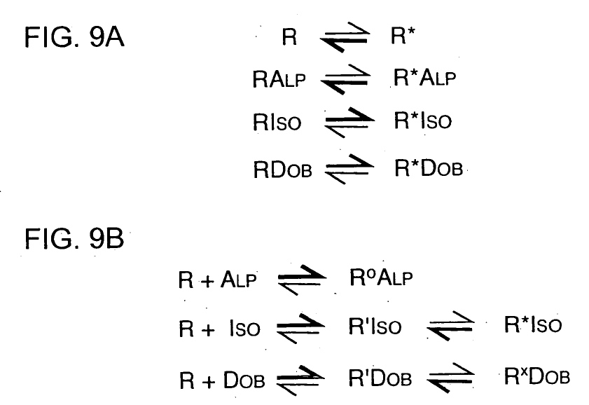

According to the prevailing two-state model of GPCR activation, receptors exist in an equilibrium between a resting (R) state and an active (R*) state which stimulates the G protein (Samama, et al. (1993) J Biol Chem 268(7), 4625-36; 30. Lefkowitz, et al. (1993) Trends Pharmacol Sci 14(8), 303-7; Leff, P. (1995) Trends Pharmacol Sci 16(3), 89-97). Agonists preferentially enrich the R* state, while inverse agonists select for the R state of the receptor. Neutral antagonists possess an equal affinity for both states and function simply as competitors. In this simple model, functional differences between drugs can be explained by their relative affinity for the single active R* state (FIG. 9A). Alternatively, differences in efficacy between drugs have been explained by ligand-specific receptor states (Kenakin, T. (1997) Trends Pharmacol Sci 18(11), 416-7; Tucek, S. (1997) Trends Pharmacol Sci 18(11), 414-6; Strange, P. G. (1999) Biochem Pharmacol 58(7), 1081-8). Our lifetime experiments can best be explained by a model with multiple agonist-specific active states (FIG. 9B).

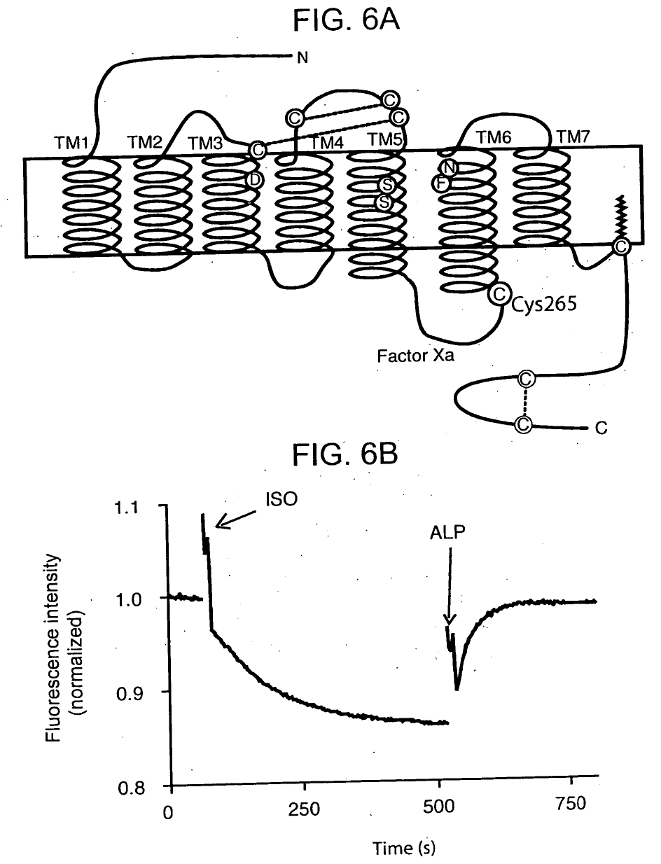

Based on these data, and without being held to theory, the inventors propose a model whereby receptor activation occurs through a sequence of conformational changes. Upon agonist binding, the receptor undergoes a conformational change to an intermediate state (R′) that is associated with a narrowing and rightward shift in the long lifetime distribution. The less abundant active state, represented by the short lifetime, is different for the full agonist ISO(R*) and the partial agonists DOB and SAL (RX). The relatively slow, temperature-dependent rate of change of fluorescence intensity following agonist binding and the rapid rate of reversal by antagonist and FIG. 6B) suggest that transitions from the intermediate state to the active state are relatively rare high energy events. It is likely that in vivo the active conformation is further stabilized by interactions between the receptor and its cognate G protein Gₛ. Thus, one might expect the proportion of receptor in the active state to be greater when the receptor is coupled with Gₛ.

### Example 10

### Modified β2-AR Having Introduced Protease Cleavage Site(s) as Conformationally Sensitive Detectable Probe

In one embodiment, the conformationally sensitive probe is a protease cleavage site introduced into the GPCR. This can be accomplished by, for example, introducing a protease cleavage site into the second or third intracellular loop of the GPCR. This is exemplified in FIG. 12, which shows the amino acid sequence of the native human β₂-adrenergic receptor and modifications that can be made within the second intracellular loop or within the third intracellular loop to insert a protease cleavage site. The protease cleavage site in this example is for the protease of the tobacco etch virus (TEV), which recognizes and cleaves at the amino acid sequence ENLYFQG (SEQ ID NO:2) between the glutamine and glycine residues.

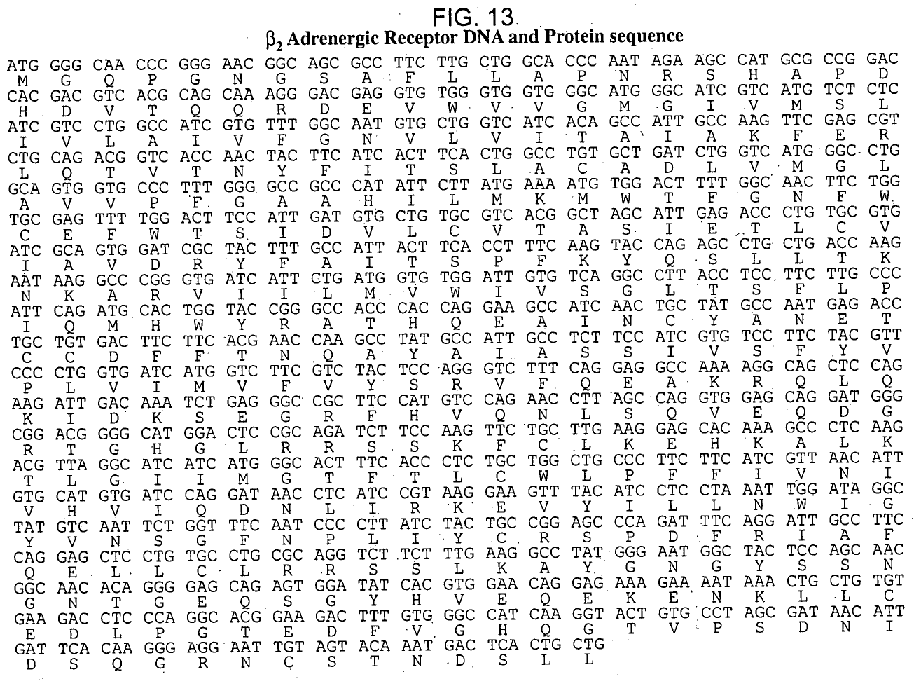

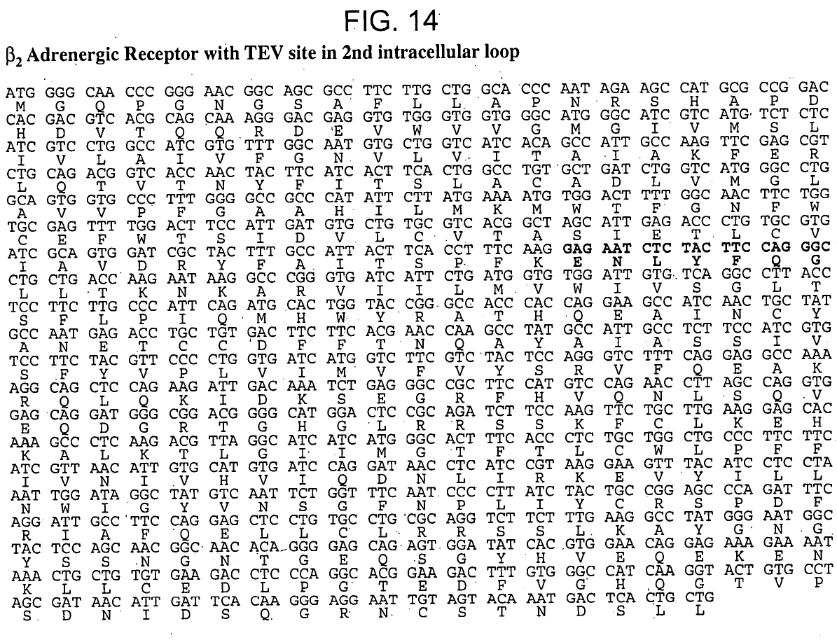

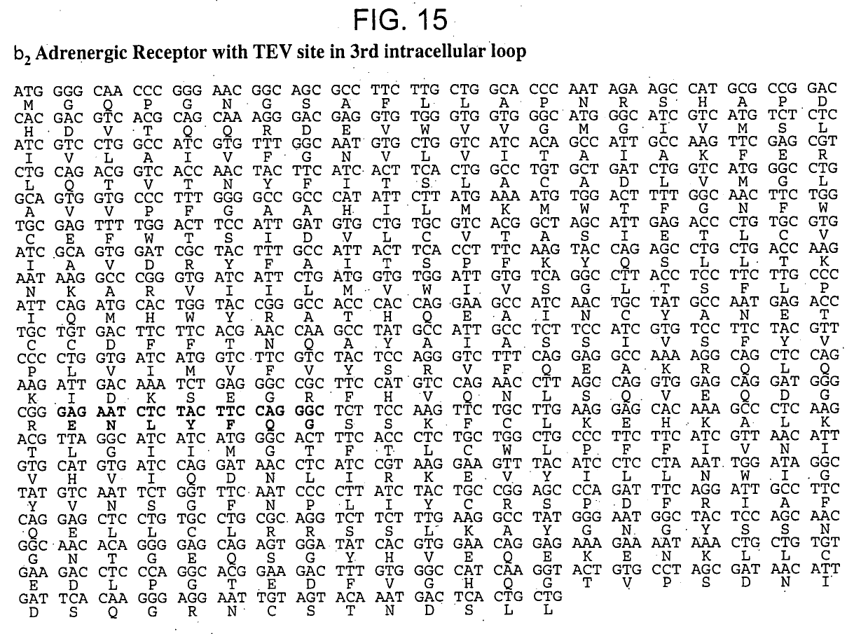

Introduction of the TEV protease cleavage site can be accomplished according to methods well known in the art. The nucleotide and amino acid sequence of native β2-AR are provided in FIG. 13. This sequence is modified to have the amino acid residues in either the second intracellular loop or the third intracellular loop as indicated in FIG. 12. A modified β2-AR having a TEV protease cleavage site in the second intracellular loop can be constructed by modifying the corresponding coding sequence as illustrated in FIG. 14. Similarly, a modified β2-AR having a TEV protease cleavage site in the third intracellular loop can be constructed by modifying the corresponding coding sequence as illustrated in FIG. 15.

### Example 11

### GPCR Having a TEV Protease Cleavage Site as a Conformationally Sensitive, Detectable Probe

The β₂ adrenergic receptor was modified to introduced a Flag epitope at the amino terminus and a TEV site within the third intracellular loop between residues 254 and 260 of the native protein (FIG. 11A). The modified β₂ adrenergic receptor was expressed in insect cells and membranes were prepared. Membranes were incubated in the presence or absence of the β₂ agonist isoproterenol for 5 minutes at 20° C. Recombinant TEV was added to the receptor and incubated for 30 minutes at 20° C. The TEV cleavage was stopped by the addition of sodium dodecyl sulfate (final concentration 1% w/v). Membrane proteins were resolved by SDS-PAGE and blotted onto nitrocellulose. Intact and cleaved β₂ adrenergic receptor was detected by probing the blot with M1 antibody.

As demonstrated in FIG. 11B and FIG. 11C, TEV cleavage of the 12 adrenergic receptor was enhanced in the presence of isoproterenol.

### Example 12

### Modified μ Opioid Receptor Having Introduced Protease Cleavage Site(s) as Conformationally Sensitive Detectable Probe

The μ opioid receptor is another example of a GPCR that can be modified to contain a protease cleavage site as a conformationally sensitive probe. The modified μ opioid receptor-can be generated by, for example, introducing a protease cleavage site into the second or third intracellular loop of the GPCR. FIG. 16 is a schematic showing the amino acid sequence of human μ-opioid receptor and modifications that can be made within the second intracellular loop or within the third intracellular loop to insert a protease cleavage site (exemplified by tobacco etch virus (TEV)) that can serve as a conformationally sensitive probe for ligand binding.

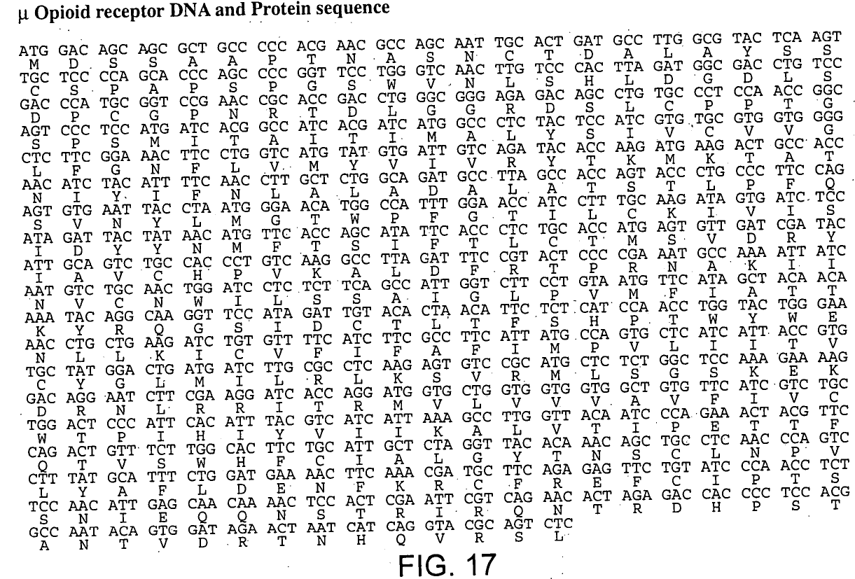

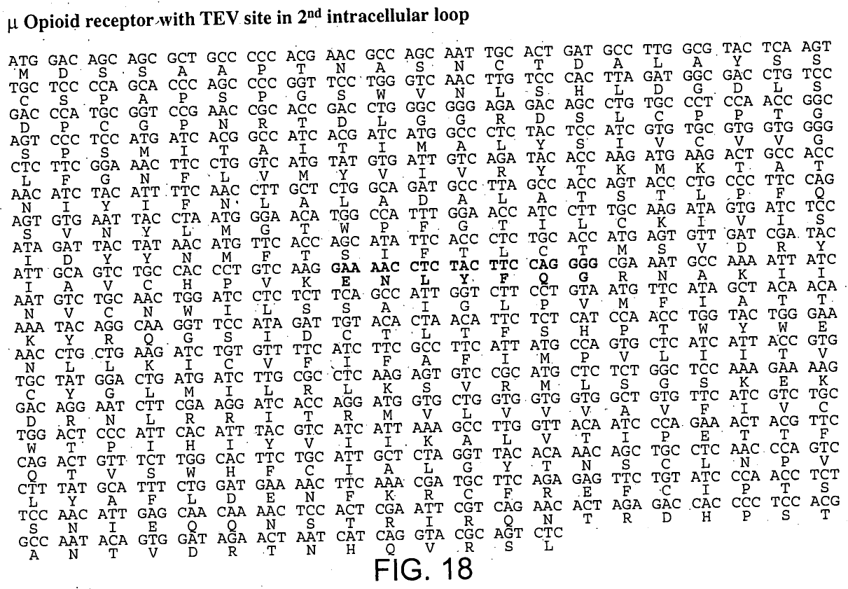

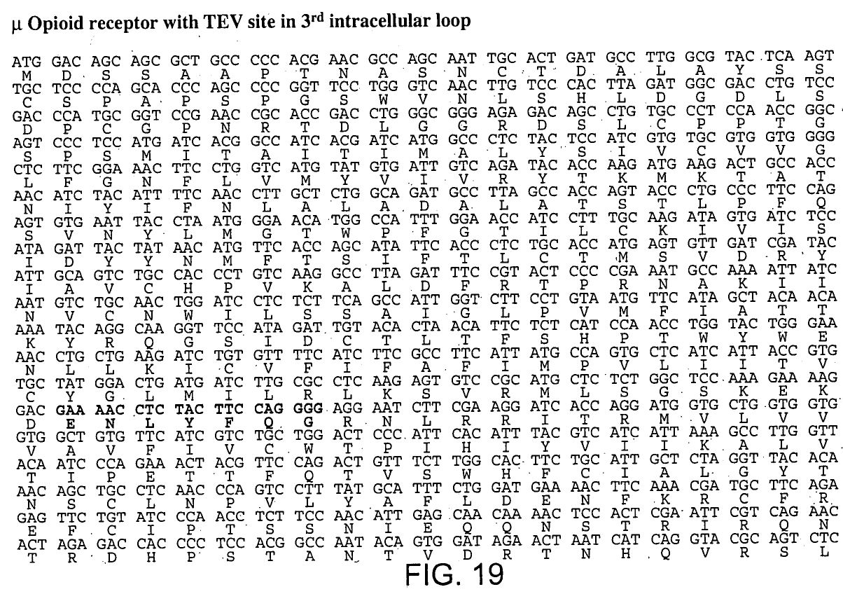

Introduction of the TEV protease cleavage site can be accomplished according to methods well known in the art. The nucleotide and amino acid sequence of native opioid receptor are provided in FIG. 17. This sequence is modified to have the amino acid residues in either the second intracellular loop or the third intracellular loop as indicated in FIG. 16. A modified μ opioid receptor a TEV protease cleavage site in the second intracellular loop can be constructed by modifying the corresponding coding sequence as illustrated in FIG. 18. Similarly, a modified μ opioid receptor having a TEV protease cleavage site in the third intracellular loop can be constructed by modifying the corresponding coding sequence as illustrated in FIG. 19.

### CONCLUSIONS

The results described above have implications for drug discovery and efforts to obtain high resolution crystal structures of MSST proteins, such as GPCRs. The results described herein indicate that these proteins are relatively plastic. The number of conformations that we observed in these experiments may represent only a few of a larger spectrum of possible conformations that could be stabilized by drugs. Thus, it may be possible to identify even more potent agonists or agonists that can alter MSST protein activity (e.g., G protein coupling specificity to a GPCR). Moreover, these results show that members of a specific class of MSST proteins (such as the GPCRs) undergo similar conformational changes upon activation.

As demonstrated above, the effect of agonists and partial agonists on the fluorescence intensity of FM-β₂AR correlates well with their biological properties. Binding of the full agonist isoproterenol to FM-β₂AR induces a conformational change that leads to a decrease in fluorescence intensity of FM bound to Cys265 by ˜15% (FIG. 6B), while binding of partial agonists results in a smaller change in intensity and binding of antagonists has no effect. Agonist-induced movement of FM bound to Cys265 was characterized by examining the interaction between the fluorescein at Cys265 and fluorescence quenching reagents localized to different molecular environments of the receptor. By site-specific labeling with a single fluorophore on the cytoplasmic extension of TM6 and with a single quencher on the cytoplasmic extension of TM5, evidence was obtained and described herein for movement of these two labeling sites toward each other. This observation and the results of studies using either an aqueous quencher or quenchers that partition into the detergent micelle are most consistent with either a clockwise rotation of TM6 and/or a tilting of the cytoplasmic end of TM6 toward TM5.

These results provide insight into the nature of the structural changes that occur upon agonist binding. Using conventional spectroscopy, no change in the fluorescence intensity from FMβ₂AR upon antagonist binding. This could indicate that antagonists do not alter receptor structure or that the structural changes are not detectable by FM bound to Cys265. However, other conformationally sensitive detectable probes placed at other positions in the protein may provide for detection of antagonist binding.

Of greater interest is the structural basis of partial agonism. Partial agonists induce a smaller change in intensity of FM-β₂AR than do full agonists. Without being held to theory, two models could explain this observation. If it is assumed that the receptor exists in two functional conformational states, inactive or active, then a partial agonist may simply induce a smaller fraction of receptors to undergo the transition to the active state than does the full agonist. Alternatively, partial agonists may induce a conformation distinct from that induced by full agonists. Conventional fluorescence spectroscopy, which represents an average intensity over a population of fluorescent molecules, does not distinguish between these two models. Fluorescence lifetime spectroscopy studies indicated that partial agonists and agonists induce distinct conformations. Moreover, structural effects of antagonist binding were observed that could not be detected by conventional spectroscopy. These results help elucidate the structural mechanisms which underlie ligand efficacy, and further aid rational drug design.

An integral detectable moiety (a TEV protease site), placed near Cys 265 of the beta 2 adrenergic also detects conformational changes upon agonist binding. We observed that TEV is more efficient at cleaving the TEV site-modified beta 2 adrenergic in the presence of an agonist. Thus, both of these two conformationally sensitive probes (fluorescein and the TEV protease site) are capable of detecting ligand-induced conformational changes.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A method for identifying an agent that modulates activity of a membrane-spanning, signal-transducing (MSST) protein, the method comprising:
contacting a membrane-spanning, signal-transducing (MSST) protein with a candidate agent, the MSST protein having a conformationally-sensitive detectable probe positioned on or within a conformationally sensitive region of the MSST protein, wherein interaction of the MSST protein with an agonist or antagonist causes a conformational change in the conformationally sensitive region and a change in a detectable signal of the conformationally sensitive detectable probe; and
detecting the detectable signal of the conformationally sensitive detectable probe resulting from said contacting;
wherein detection of a change in a level of the detectable signal in the presence of the candidate agent relative to a control level of detectable signal indicates the candidate agent modulates activity of the MSST protein.

2. The method of claim 1, wherein the conformationally-sensitive detectable probe is a detectable chemical label attached to an amino acid residue of the conformationally sensitive region.

3. The method of claim 1, wherein the conformationally-sensitive detectable probe is a protease cleavage site and the detectable signal is a protease cleavage product.

4. The method of claim 1, wherein the conformationally-sensitive detectable probe comprises two protease cleavage sites, which cleavage sites flank a detectable polypeptide so that cleavage of the cleavage sites results in release of the detectable polypeptide, and wherein the detectable signal is the detectable polypeptide.

5. The method of claim 1, wherein the conformationally-sensitive detectable probe is an immunodetectable epitope and the detectable signal is present on a primary antibody that specifically binds the epitope or on a secondary antibody that specifically binds the primary antibody.

6. The method of claim 1, wherein the conformationally sensitive region is in an intracellular loop, an extracellular loop, an N-terminal domain, or a C-terminal domain of the MSST protein.

7. The method of any one of claims 1-6, wherein the MSST protein is selected from the group consisting of a G protein coupled receptor (GPCR), an ion channel, or a transporter protein.

8. The method of claim 1, wherein the MSST protein is a G-protein coupled receptor (GPCR), and the conformationally sensitive region is an intracellular loop, an extracellular loop, an N-terminal domain, or a C-terminal domain of the GPCR.

9. The method of claim 8, wherein the conformationally sensitive region is a third intracellular loop of the GPCR, and the conformationally sensitive detectable probe is a detectable chemical label attached to one or more amino acid residues within the third intracellular loop so that a conformational change in the GPCR due to interaction with an agonist or antagonist causes a change in the detectable signal of the detectable probe.

10. The method of claim 9, wherein the detectable chemical label is attached to an amino acid residue corresponding to amino acid residue at position 265 in a β2-adrenergic receptor.

11. The method of claim 8, wherein the conformationally sensitive detectable probe is a protease cleavage site and the detectable signal is a protease cleavage product.

12. The method of claim 11, wherein the protease cleavage product is an N-terminal fragment of the GPCR, a C-terminal fragment of the GPCR.

13. An apparatus for detecting a molecule that modulates activity of a membrane-spanning, signal-transducing protein, the apparatus comprising:
a membrane-spanning, signal-transducing protein (MSST) of any one of claims 1-12; and
a immobilization phase to which the MSST protein is attached.

14. A kit for use in screening a candidate agent, the kit comprising:
a membrane-spanning, signal-transducing protein (MSST) of any one of claims 1-12.

15. The kit of claim 14, wherein the MSST protein is attached to an immobilization phase.


SEQUENCE LISTING (22 sequences)

SEQ ID NO: 1 - protein, 8 aa, Artificial Sequence
  epitope tag peptide
DYKDDDDK

SEQ ID NO: 2 - protein, 8 aa, Artificial Sequence
  epitope tag peptide
DYKDEDDK

SEQ ID NO: 3 - protein, 9 aa, Artificial Sequence
  epitope tag peptide
AWRHPQFGG

SEQ ID NO: 4 - protein, 13 aa, Artificial Sequence
  epitope tag peptide
YPYDVPDYAIEGR

SEQ ID NO: 5 - DNA, 1239 bp, Homo sapiens
  CDS (1)...(1239)
atggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggctagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagctcctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctg

SEQ ID NO: 6 - protein, 413 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 7 - DNA, 1239 bp, Artificial Sequence
  Beta-2 Adrenergic Receptor with TEV site in 2nd intracellular loop
atggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggctagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactcgaaaacctctacttccaggggttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagctcctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctg

SEQ ID NO: 8 - protein, 413 aa, Artificial Sequence
  Beta-2 Adrenergic Receptor with TEV site in 2nd intracellular loop
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLENLYFQGLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 9 - DNA, 1251 bp, Artificial Sequence
  Beta-2 Adrenergic Receptor with TEV site in 3rd intracellular loop
atggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggctagcattgagaccctgtgcgtgatcgcagtggatcgctactttgccattacttcacctttcaaggagaatctctacttccagggcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagctcctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctg

SEQ ID NO: 10 - protein, 417 aa, Artificial Sequence
  Beta-2 Adrenergic Receptor with TEV site in 3rd intracellular loop
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKENLYFQGLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 11 - DNA, 1176 bp, homo sapiens
  CDS (1)...(1176)
atggacagcagcgctgcccccacgaacgccagcaattgcactgatgccttggcgtactcaagttgctccccagcacccagccccggttcctgggtcaacttgtcccacttagatggcgacctgtccgacccatgcggtccgaaccgcaccgacctgggcgggagagacagcctgtgccctccaaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatagctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcaggtacgcagtctc

SEQ ID NO: 12 - protein, 392 aa, homo sapiens [sequence omitted]

SEQ ID NO: 13 - DNA, 1176 bp, Artificial Sequence
  ′ Opioid receptor with TEV site in 2nd intracellular loop
atggacagcagcgctgcccccacgaacgccagcaattgcactgatgccttggcgtactcaagttgctccccagcacccagccccggttcctgggtcaacttgtcccacttagatggcgacctgtccgacccatgcggtccgaaccgcaccgacctgggcgggagagacagcctgtgccctccaaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggaaaacctctacttccaggggcgaaatgccaaaattatcaatgtctgcaactggatcctctcttcagccattggtcttcctgtaatgttcatagctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcaggtacgcagtctc

SEQ ID NO: 14 - protein, 392 aa, Artificial Sequence
  ′ Opioid receptor with TEV site in 2nd intracellular loop
MDSSAAPTNASNCTDALAYSSCSPAPSPGSWVNLSHLDGDLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKENLYFQGRNAKIINVCNWILSSAIGLPVMFIATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQVRSL

SEQ ID NO: 15 - DNA, 1197 bp, Artificial Sequence
  ′ Opioid receptor with TEV site in 3rd intracellular loop
atggacagcagcgctgcccccacgaacgccagcaattgcactgatgccttggcgtactcaagttgctccccagcacccagccccggttcctgggtcaacttgtcccacttagatggcgacctgtccgacccatgcggtccgaaccgcaccgacctgggcgggagagacagcctgtgccctccaaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatagctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacgaaaacctctacttccaggggaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcaggtacgcagtctc

SEQ ID NO: 16 - protein, 399 aa, Artificial Sequence
  ′ Opioid receptor with TEV site in 3rd intracellular loop
MDSSAAPTNASNCTDALAYSSCSPAPSPGSWVNLSHLDGDLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFIATTKYRQGSIDCTLTFSHPTWYWENLLKICVFIFAFIMPVLIITVCYGLMILRLKSVRMLSGSKEKDENLYFQGRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQVRSL

SEQ ID NO: 17 - protein, 10 aa, Artificial Sequence
  Hemagglutinin tag
CYPYDVPDYA

SEQ ID NO: 18 - protein, 11 aa, Artificial Sequence
  c-myc tag
CEQKLISEEDL

SEQ ID NO: 19 - protein, 5 aa, Artificial Sequence
  enterokinase cleavage site
DDDDK

SEQ ID NO: 20 - protein, 4 aa, Artificial Sequence
  factor Xa cleavage site
IEGR

SEQ ID NO: 21 - protein, 6 aa, Artificial Sequence
  thrombin cleavage site
LVPAGS

SEQ ID NO: 22 - protein, 8 aa, Artificial Sequence
  renin cleavage site
HPFHLVIH